(19) United States
(12) Patent Application Publication
(10) Pub. No.: US 2025/0352560 A1
(43) Pub. Date: Nov. 20, 2025

(54) CANNABINOID-BASED COMPOSITIONS, PRODUCTS, AND METHODS FOR SUPPORTIVE CARE

(71) Applicant: EO Care, Inc., Boston, MA (US)

(72) Inventors: David C. Batista, Brookline, MA (US); Stacey Batista, Brookline, MA (US); Benjamin P. Caplan, Needham, MA (US); Sean J. Collins, Wellesley, MA (US); Nial C. DeMena, Worcester, MA (US); Emily R. Hajjar, Garnet Valley, PA (US); Birch Norton, Ipswich, MA (US); Kristen P. Parton, Medfield, MA (US); Christine R. Pillsbury, Danvers, MA (US); Geordie McClelland, Cambridge, MA (US); Brooke K. Worster, Philadelphia, PA (US)

(21) Appl. No.: 19/209,783

(22) Filed: May 15, 2025

Related U.S. Application Data
(60) Provisional application No. 63/648,037, filed on May 15, 2024.

(51) Int. Cl. A61K 31/00; A61P 1/08; A61P 25/20; A61P 25/22; A61P 29/00; G16H 10/60; G16H 20/90

(52) U.S. Cl. CPC A61K 31/658; A61P 1/08; A61P 25/20; A61P 25/22; A61P 29/00; G16H 10/60; G16H 20/90

(57) ABSTRACT

Disclosed herein, inter alia, are methods of improving an outcome of a subject, the methods comprising: administering a composition comprising a cannabinoid to the subject based on a treatment plan that is based on the subject's data, the cannabinoid intended to cause an improved outcome of the subject, wherein the outcome comprises one or more of: (i) reducing a condition of the subject, (ii) improving mobility of the subject and reducing difficulty of performing general activities by the subject, (iii) reducing difficulty of performing daily living activities by the subject, or (iv) reducing difficulty of performing activities related to work, social, or leisure time by the subject.

### RELATED APPLICATIONS

This application claims the benefit of U.S. Provisional Application No. 63/648,037, filed on May 15, 2024. The entire teachings of the above application(s) are incorporated herein by reference.

### BACKGROUND

The use of cannabinoids for medical purposes, especially among cancer patients, has seen a significant increase in recent years. However, concerns have arisen due to the prevalent use of smoked cannabis and the associated risk of cannabis use disorder. Additionally, there is a lack of products incorporating cannabinoids tailored specifically for cancer patients, who face unique challenges related to the disease and its treatment.

Cancer patients often experience difficulties such as loss of appetite, weight loss, and chronic pain, requiring effective therapeutic interventions. While cannabis shows promise as a potential remedy, existing products often involve combustion-based delivery methods, lack standardized dosing, and fail to address the specific needs of individuals undergoing cancer treatments. Furthermore, concerns arise regarding potential drug interactions and polypharmacy issues when using these products alongside conventional cancer therapies.

Statistics indicate that a significant proportion of cancer patients, estimated at 40% to 50%, seek relief from debilitating symptoms through cannabis use. Thus, there is a pressing need to develop medicalized products containing cannabinoids, carefully crafted to meet the specific requirements of this patient population, as well as a guidance system for using the products.

### SUMMARY

Cannabinoids have also emerged as a potential solution for the aging population due to their analgesic and anti-inflammatory properties. The aging population often faces challenges associated with chronic pain and inflammation, impacting their quality of life. Further, while cannabinoid-based products have emerged as promising treatments for chronic pain, cancer-related symptoms, aging-related challenges, sleep disturbances, and anxiety, their effectiveness may vary due to individual differences and inconsistent dosing. Moreover, the absence of personalized care regimens tailored to individual needs limits their therapeutic potential.

Disclosed herein are cannabinoid-based products that are safe, effective, and tailored to a patient's requirements. In some embodiments, the cannabinoid-based products are enhanced by a digital platform (e.g., a telehealth platform comprising a model-based care regimen generator) which leverages advanced algorithms and patient data to optimize treatment protocols and maximizes the therapeutic effects of cannabinoid therapy across various health conditions.

In some embodiments, the disclosure provides a method of improving an outcome of a subject, the method comprising: administering a composition comprising a cannabinoid to the subject based on a treatment plan that is based on the subject's data, the cannabinoid intended to cause an improved outcome of the subject, wherein the outcome comprises one or more of: (i) reducing a condition of the subject, (ii) improving mobility of the subject and reducing difficulty of performing general activities by the subject, (iii) reducing difficulty of performing daily living activities by the subject, or (iv) reducing difficulty of performing activities related to work, social, or leisure time by the subject.

In some embodiments, the disclosure provides a method of reducing pain, sleep disturbance, anxiety, or gastrointestinal nausea and vomiting in a subject, the method comprising: administering a composition comprising a cannabinoid to the subject.

Also disclosed herein are compositions comprising a first cannabinoid, a second cannabinoid, and an agent. In some embodiments, the agent comprises protein, amino acids, or a combination thereof.

In some embodiments, the disclosure provides a delivery system comprising a composition of the present disclosure. In some embodiments, the delivery system comprises a micro-pelletized capsule.

### BRIEF DESCRIPTION OF THE DRAWINGS

The foregoing will be apparent from the following more particular description of example embodiments, including those illustrated in the drawings interspersed herein. The drawings are not necessarily to scale, emphasis instead being placed upon illustrating embodiments.

FIG. 1 shows an example platform where a subject can review a treatment plan and products, order the products, schedule a pickup, and begin treatment plan.

FIG. 2 shows an example platform which provides shipping information to a subject.

FIG. 3 shows example products which are being shipped and products that may be picked up.

FIG. 4 shows example instructions for using a product of the present disclosure.

FIG. 5 shows example instructions for using a product of the present disclosure.

FIG. 6 shows example treatment plan for a subject.

FIG. 7 shows the PROMIS® (Patient-Reported Outcomes Measurement Information System) metric scores for a four-week patient cohort.

### DETAILED DESCRIPTION

A description of example embodiments follows.

The needs of various population age groups (e.g., adults) may be addressed by providing cannabinoid-based products tailored to alleviate various symptoms and conditions, such as chronic pain and inflammation associated with aging. These products may utilize precise formulations, advanced delivery methods, and digital integration to ensure safety, efficacy, and ease of use for consumers, such as older adult consumers.

1. Formulation: In some embodiments, product formulations comprise cannabinoids, functional ingredients, and nutritional supplements in ratios optimized for addressing chronic pain, inflammation, cognitive decline, and sleep disturbances in older adults. Novel combinations of compounds are identified to target age-related symptoms while minimizing potential adverse effects.

In some embodiments, cannabinoid-based products are formulated to target specific symptoms and conditions. These products may incorporate cannabinoids, advanced functional ingredients, and nutritional supplements in precise formulations optimized for therapeutic efficacy. For example, by selecting the most suitable cannabinoid-based products for each patient's needs, the product helps to ensure targeted and effective treatment.

Formulation: In some embodiments, product formulations that combine cannabinoids, functional ingredients, and nutritional supplements may target specific symptoms (e.g., sexual health, joint pain, neuropathy, PTSD, spasticity, seizures) and side effects experienced by patients (e.g., cancer patients).

Non-Combustible Delivery: Unlike traditional cannabis products that rely on combustion-based delivery methods such as smoking or vaping, products of the present disclosure may be delivered in non-combustible form factors. This not only eliminates the risks associated with inhaling smoke but also ensures consistent dosing and precise delivery of active ingredients. In addition to non-combustible form factors such as topicals, tinctures, and oral formulations, products of the present disclosure include delivery systems and are not limited to transdermal patches, tablets (e.g., sublingual tablets, orally dissolvable tablets), gummies, snacks, meal replacement (e.g., shakes, smoothies, bars, powders, pre-packaged meals), beverages, and capsules (e.g., controlled-release capsules, micro-pelletized capsules). These dosage forms and methods ensure precise dosing, sustained release of active ingredients, and enhanced bioavailability, maximizing therapeutic efficacy for older adults.

Safety and Efficacy: Product formulation processes may adhere to quality control and regulatory compliance. Each product batch may undergo rigorous testing to verify potency, purity, and absence of contaminants, helping to ensure safe and reliable products for cancer patients.

2. Tailored Health Outcomes: The present disclosure relates to, among other things, achieving specific health outcomes relevant to older adults, including pain relief, reduced anxiety, improved mobility, cognitive enhancement, and enhanced sleep quality. Products disclosed and contemplated herein may target these outcomes and address the challenges faced by older adults dealing with chronic pain and inflammation. By targeting health outcomes, products of the present disclosure may be used to address challenges faced by cancer patients, including but not limited to impaired appetite, weight loss, chronic pain, nausea, and insomnia.

3. Advanced Delivery Methods: In addition to non-combustible form factors such as but not limited to topicals, tinctures, aerosol formulations, and oral formulations, products of the present disclosure may utilize delivery systems such as but not limited to transdermal patches, inhalers, beverages, and controlled-release capsules. These delivery methods ensure precise dosing, sustained release of active ingredients, and enhanced bioavailability, maximizing therapeutic efficacy for older adults.

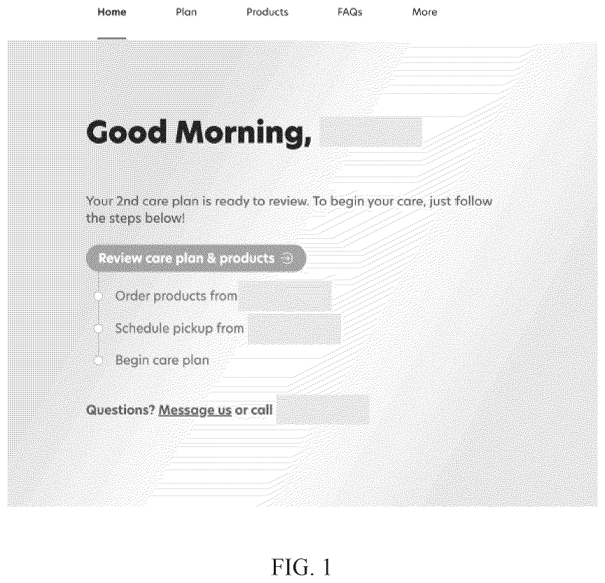

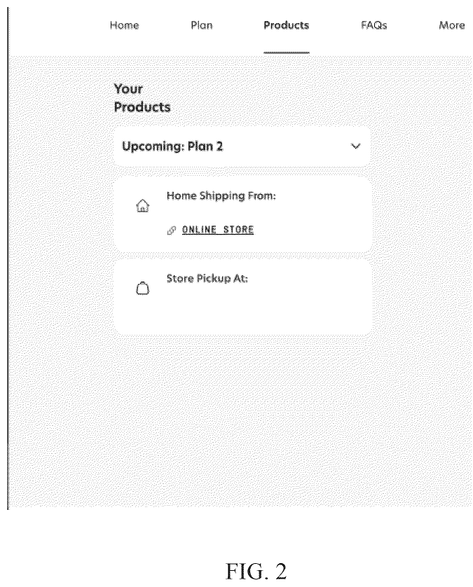

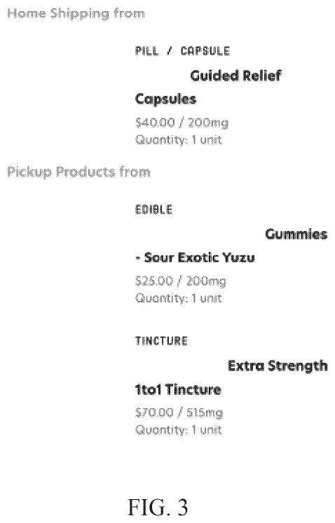

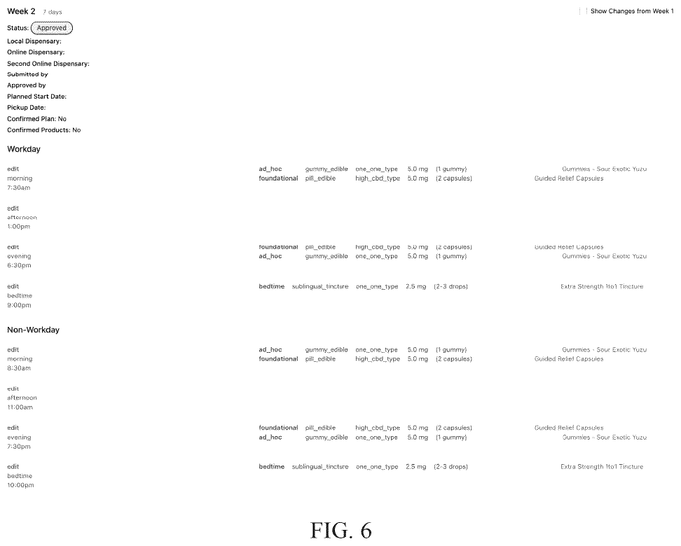

4. Enhanced Digital Integration: In addition to formulation and delivery methods, a model-driven personalized care regimen (see FIGS. 1-6) through advanced digital integration (e.g., digital platform) can, in some embodiments, be incorporated in the methods and products (e.g., composition, meal replacement, snack, beverage, device, etc.) of the present disclosure. For example, using a digital platform, a subject may access personalized regimens and dosing instructions (FIG. 6) tailored to their specific needs and medical history. This digital integration not only enhances patient convenience but may also enable real-time monitoring and adjustment of treatment plans based on individual responses and feedback.

The patient digital platform can, for example, utilize artificial intelligence (AI) algorithms to analyze patient data, including but not limited to medical history, genetic predispositions, biomarker profiles, and real-time health metrics (e.g., sleep, pain, anxiety, nausea/appetite PROMIS® metrics, metrics around sexual health, overall well-being, and seizures). Data from both patients and users without any treatment plan may be integrated into the digital platform to optimize performance.

In some embodiments, patient digital platform may be utilized for product optimization. For example, the platform may gather real-time data on treatment outcomes, adherence, and patient-reported experiences, thereby informing product refinement, identifying any unmet needs, and supporting data-driven decisions (e.g., for optimizing therapeutic efficacy and user experience).

5. Model-Driven Personalized Care Regimens: In some embodiments, complementing the cannabinoid-based products is a model-based treatment plan generator that utilizes advanced algorithms to analyze patient data and generate personalized treatment plans. The regimens can outline specific combinations of cannabinoid-based products, dosing schedules, and administration methods tailored to individual patient characteristics and treatment goals.

Patient data, including but not limited to symptom severity, treatment responses, and adherence, may be continuously or continually monitored and analyzed to refine treatment protocols over time. By leveraging real-time feedback and predictive analytics, the generator identifies trends, patterns, and potential interventions to further enhance therapeutic efficacy and patient satisfaction.

Through the model-based treatment plan generator (e.g., model-based care regimen generator), patients receive personalized treatment protocols that optimize the therapeutic effects of cannabinoid therapy. These tailored protocols consider individual health profiles, symptom severity, and treatment responses, ensuring precision and effectiveness in cannabinoid-based treatment.

In some embodiments, model-driven personalized care regimens help address the individual needs of older adults. By leveraging AI and machine learning techniques, digital platforms generate dynamic treatment plans that adapt in response to changing patient conditions, treatment responses, and emerging research findings. For non-limiting example, the platform leverages large language models (LLMs) to personalize care by generating treatment plans tailored to individual patients. LLMs may be prompted with patient-specific data (e.g., age, symptoms, medical history) to generate treatment plans and clarify reasoning behind model predictions, enabling transparent and personalized care. In another example, the platform may receive a treatment plan (e.g., a personalized care plan) and explain the plan to a user, in order to help the patient understand their treatment goals, medications, and lifestyle recommendations.

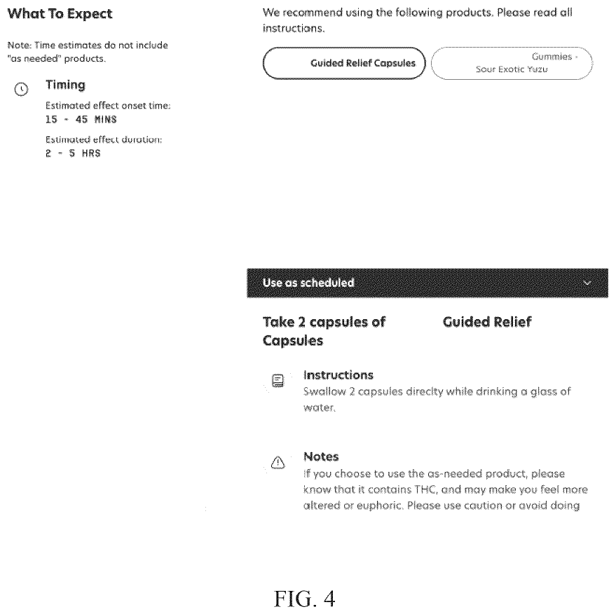

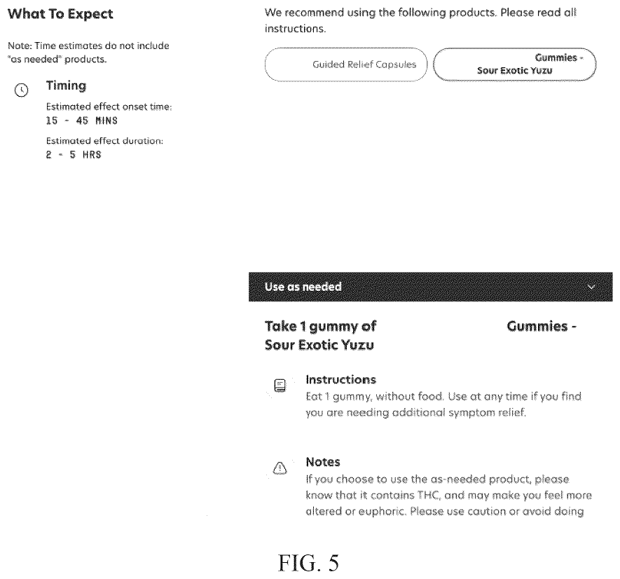

6. Integration with Product Formulation: The personalized care regimens generated by the digital platform can be integrated with cannabinoid-based products of the present disclosure (e.g., FIGS. 4-5). Product formulation can be optimized to complement the specific therapeutic goals and dosing requirements identified through the personalized care regimen. The integration can help to promote efficacy and safety while reducing the risk of adverse events or suboptimal outcomes.

7. Real-Time Monitoring and Feedback Loop: Through continuous monitoring of patient data and feedback mechanisms, digital platforms enable proactive intervention and optimization of treatment outcomes. Clinicians can remotely access real-time health metrics and patient-reported outcomes, allowing for timely adjustments to treatment plans and personalized interventions.

The present disclosure relates to, among other things, combinations of cannabinoids, functional ingredients, and nutritional supplements to achieve specific health outcomes, as measured by PROMIS® (Patient-Reported Outcomes Measurement Information System) metrics, Patient Identified Outcomes (PIOs), and other medical indicators. Products of the present disclosure may be delivered in non-combustible form factors (e.g., beverages, powders) using precise, pre-measured dosing mechanisms, in contrast to less controlled delivery methods such as smoking, vaping, or dispensary tinctures administered via droppers, thereby supporting consistent administration and improved safety and efficacy. A patient digital platform may help to create personalized regimens and dosing instructions, optimizing the impact of the cannabinoid-based products on cancer patients' well-being.

The present disclosure also relates to, among other things, cannabinoid-based products specifically formulated to address chronic pain, cancer-related symptoms, aging-related challenges, sleep disturbances, and anxiety. These products may be enhanced by a model-based care regimen generator that tailors treatment protocols to individual patient characteristics and treatment goals, optimizing therapeutic efficacy.

In some embodiments, data obtained from patients (e.g., patient use, responses, and outcomes) are analyzed to inform subsequent iterations of a product, including but not limited to modifications to formulation, dosage, and/or mode of administration. In some embodiments, such data-driven refinements enable ongoing optimization of a product's safety, efficacy, and usability.

In some embodiments, the present disclosure relates to cannabinoid-based therapeutics (e.g., as disclosed elsewhere herein) for various subjects (e.g., older adults dealing with chronic pain and inflammation, cancer patients). Formulation techniques, delivery methods (e.g., non-combustible delivery methods), digital platform integration, and model-driven personalized care regimens may be implemented in order to address the needs of the subjects, maximize therapeutic benefits, and improve patient outcomes.

In some embodiments, one or more compositions, meal replacement or snack, beverage (e.g., a shake), or device of the present disclosure may be used in conjunction with a digital platform (e.g., telehealth platform) which provides, among other things, recommendations for cannabis-based treatment to an individual.

Additional embodiments and details of the digital platform may be found in U.S. patent application Ser. No. 17/888,260, the contents of which are incorporated herein by reference in its entirety.

### Compositions

The present disclosure relates generally to compositions (e.g., pharmaceutical compositions) comprising a first cannabinoid and/or an agent. In some embodiments, the first cannabinoid is cannabigerol, cannabidiol, cannabinol, cannabichromene, cannabidivarin, or a combination thereof. In some embodiments, the first cannabinoid is cannabigerol, cannabidiol, cannabinol, cannabichromene, or a combination thereof. In some embodiments, the first cannabinoid is delta-9-tetrahydrocannabinol, delta-8-tetrahydrocannabinol, tetrahydrocannabinoic acid, tetrahydrocannabivarin, or a combination thereof. In some embodiments, the first cannabinoid is tetrahydrocannabinol, tetrahydrocannabivarin, or a combination thereof. In some embodiments, the first cannabinoid is cannabidiol.

In some embodiments, compositions of the present disclosure further comprises a second cannabinoid. In some embodiments, the second cannabinoid is cannabigerol, cannabidiol, cannabinol, cannabichromene, cannabidivarin, or a combination thereof. In some embodiments, the second cannabinoid is delta-9-tetrahydrocannabinol, delta-8-tetrahydrocannabinol, tetrahydrocannabinoic acid, tetrahydrocannabivarin, or a combination thereof. In some embodiments, the second cannabinoid is cannabigerol, cannabichromene, cannabinol, delta-8-tetrahydrocannabinol, tetrahydrocannabinolic acid, cannabidiolic acid, or a combination thereof. In some embodiments, the second cannabinoid is delta-9-tetrahydrocannabinol. In some embodiments, the second cannabinoid is tetrahydrocannabivarin.

In some embodiments, a composition is a first composition and a cannabinoid is a first cannabinoid.

In some embodiments, the compositions comprise a therapeutically and/or prophylactically effective amount of a first cannabinoid, a second cannabinoid, and/or an agent. In some embodiments, a composition comprises tetrahydrocannabivarin and cannabidiol.

In some embodiments, the agent is a therapeutic agent (e.g., a peptide, a nucleic acid, a cannabinoid, a small-molecule inhibitor, isomers (e.g., stereoisomers), tautomers and salts thereof), an adjuvant, or a combination thereof. In some embodiments, the agent comprises protein, amino acids, or a combination thereof. In some embodiments, the amino acids are branched-chain amino acids. As used herein, the term “adjuvant” is defined as an additive that enhances the functional benefit of the composition to a subject in need thereof. For example, an adjuvant can provide nourishment, increased appetite and/or relaxation to the subject.

Examples of functional benefits include, but are not limited to, sustained steady energy, improving vascular health, reducing inflammation, enhancing immune system, improving cognitive function, reducing symptoms associated with aging, or improving metabolism, or a combination thereof. The nutritional composition or the nutritional combination can improve, or alleviate a condition or disease, or a plurality of conditions or diseases, including, but not limited to, neurological conditions or diseases, inflammatory conditions or diseases, and metabolic conditions or diseases.

Examples of agents include, but are not limited to, protein, amino acids, fiber, vitamins, minerals, antioxidants, flavorants, co-factors, such as enzymes (e.g., proteases), fats, carbohydrates, and sugar substitutes. In some embodiments, the agent comprises protein, amino acids, or a combination thereof.

Examples of cannabinoids include but are not limited to delta-9-tetrahydrocannabinol (49-THC), delta-8-tetrahydrocannabinol (48-THC), tetrahydrocannabinoic acid (THCA), cannabidiol (CBD), cannabidiolic acid (CBDA), cannabinol (CBN), cannabigerol (CBG), tetrahydrocannabivarin (THCV), cannabigerolic acid (CBGA), cannabichromene (CBC), isomers (e.g., stereoisomers), tautomers, salts, and combinations thereof. In some embodiments, the first cannabinoid is cannabigerol, cannabidiol, or a combination thereof. In some embodiments, the first cannabinoid is cannabigerol. In some embodiments, the second cannabinoid is tetrahydrocannabinol (e.g., 49-THC, 48-THC, or a mixture thereof).

One or more of the first cannabinoid, a second cannabinoid, and an agent may be derived from natural sources (e.g., plant sources), or artificial sources. For example, the first cannabinoid and the second cannabinoid are derived from plants of the Cannabis genus. In another example, the agent (e.g., protein) source is plant-based, such as from peas, chia seeds, rice, brown rice, fava beans, all or any combination of these.

Compositions of the present disclosure can further comprise one or more of terpenes, terpenoids, flavonoids, isomers (e.g., stereoisomers), tautomers, salts, and combinations thereof. In some embodiments, compositions of the present disclosure further comprises a terpene, a terpenoid, or a combination thereof.

An advantage of compositions of the disclosure is that they can also be customized for a particular functional benefit or personalized according to the end-user's needs and desired functional benefits, such as by adding additional agents or ingredients.

In some embodiments, the composition has a mass ratio of the first cannabinoid to the second cannabinoid that equals or exceeds about 5:1 (e.g., about 5:1, 10:1, 15:1, 20:1, 25:1, 30:1, 35:1, 40:1, 45:1, 50:1, etc.). In some embodiments, the composition has a mass ratio of the first cannabinoid to the second cannabinoid that is between about 5:1 and about 100:1 (e.g., between about 10:1 and about 100:1, between about 10:1 and about 50:1, between about 20:1 and about 50:1, between about 30:1 and about 50:1, etc.). In some embodiments, the composition has a mass ratio of the first cannabinoid to the second cannabinoid that is about 40:1. Other ratios of the first cannabinoid to the second cannabinoid are contemplated and such ratios can be ascertained by the formulator based on the subject's needs and desired functional benefits of the composition. In some embodiments, the first cannabinoid is cannabigerol, cannabidiol, or a combination thereof. In some embodiments, the second cannabinoid is tetrahydrocannabinol (e.g., delta-9-tetrahydrocannabinol). In some embodiments, the first cannabinoid is cannabigerol and the second cannabinoid is tetrahydrocannabinol. In other embodiments, the first cannabinoid is cannabidiol and the second cannabinoid is tetrahydrocannabinol.

In some embodiments, the composition has a mass ratio of the combined mass of the first cannabinoid and the second cannabinoid to the agent that exceeds about 1:100 (e.g., 1:50, 1:10, 1:5, 1:4, 1:3.5, 1:3, 1:2.5, 1:2, 1:1.5, 1:1, etc.). In some embodiments, the composition has a mass ratio of the combined mass of the first cannabinoid and the second cannabinoid to the agent that is between about 1:100 and about 1:1 (e.g., between about 1:50 and about 1:1, between about 1:10 and about 1:1, between about 1:5 and about 1:1, etc.). In some embodiments, the composition has a mass ratio of the combined mass of the first cannabinoid and the second cannabinoid to the agent that is from about 1:3 to about 1:2. Other ratios of the combined mass of the first cannabinoid and the second cannabinoid to the agent are contemplated and such ratios can be ascertained by the formulator based on the subject's needs and desired functional benefits of the composition.

In some embodiments, the agent comprises protein and amino acids, and the composition has a mass ratio of the protein to the amino acids that is from about 1:10 to about 1:50 (e.g., about 1:10 to about 1:50, about 1:10 to about 1:40, about 1:10 to about 1:30, about 1:10 to about 1:20, about 1:15 to about 1:20). Other ratios of the protein to the amino acids are contemplated and such ratios can be ascertained by the formulator based on a subject's needs and desired functional benefits of the composition.

In some embodiments, a composition comprises an agent (e.g., protein, amino acids, ctc.) in an amount of from about 1 mg to about 100 mg (e.g., about 1 mg to about 50 mg, about 10 mg to about 50 mg, about 10 mg to about 40 mg, about 10 mg to about 30 mg, about 15 g to about 30 mg, etc.).

In some embodiments, the first cannabinoid or the second cannabinoid is present in an amount of from about 1 mg to about 250 mg (e.g., about 10 mg to about 200 mg, about 20 mg to about 100 mg, about 10 mg to about 50 mg, about 2 mg to about 20 mg, about 200 mg to about 250 mg, etc.). For example, in some embodiments, a composition comprises delta-9-tetrahydrocannabinol present in an amount of about 200 mg to about 250 mg.

In some embodiments, the composition comprises 2.5 mg THC, 100 mg CBG, protein (15 mg to 30 mg), and 250 mg branched-chain amino acids.

In some embodiments, the first cannabinoid, the second cannabinoid or a combination thereof is delivered to a subject or released in a subject (after intake of the composition) over a time period of from about 1 hour to about 48 hours (e.g., about 1 hour to about 24 hours, about 2 hour to about 24 hours, about 3 hours to about 24 hours, about 4 hours to about 24 hours, about 5 hours to about 24 hours, about 6 hours to about 24 hours, about 7 hours to about 24 hours, about 8 hours to about 24 hours, etc.). For example, in some embodiments, delta-9-tetrahydrocannabinol is delivered to a subject over a time period of from about 8 hours to about 24 hours.

As described herein, the compositions can provide the indicated amounts or ratios of ingredients in various forms, such as in the form of a drinkable beverage or powder to be dissolved in water, milk, or other liquid (or slurried with a solid such as yogurt, ice cream, or other ingredients or blended). The composition can be administered in a single serving format (e.g., bottle providing a single dose of the preceding ingredients) or in single doses of powder or solid. In other embodiments, labeling or other packaging or instruction is provided so that the composition can be provided in doses or aliquots according to measurement, e.g., as in a multi-serve bottle or in the form of a powder or other solid that be dosed (e.g., by using a provided scoop) so as to provide the indicated total ingredient amounts.

Compositions of the present disclosure may further comprise an excipient (e.g., a pharmaceutically acceptable excipient). Non-limiting examples of excipients include inert diluents, flavoring agent, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition. In some embodiments, the excipient comprises a flavoring agent.

Examples of diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Examples of granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Examples of surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monoolcate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), dicthylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Examples of binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Examples of preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Examples of antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Examples of chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Examples of antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Examples of antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Examples of alcohol preservatives include ethanol, polyethylene glycol, phenol, phenolic compounds, bisphenol, chlorobutanol, hydroxybenzoate, and phenylethyl alcohol.

Examples of acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Examples of buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Examples of lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Examples of natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Example synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

### Modes of Delivery, Packaging

The present disclosure relates to compositions that may be modular/component systems of prepared or unprepared food such as, but not limited to, meal replacements, bars, bites, drinks, snacks, meals, desserts, cereals, salad, side dish, sauces, desserts, spreads etc. For example, the compositions can used in prepared or unprepared foods such that consumers can select a specific food or drink item, such as a bottle of juice, bar, a salad, a meal knowing that the composition will keep them in a safe metabolic range. In certain embodiments, such compositions are packaged and marked for a particular cobort or population.

In other embodiments, the present disclosure relates to a meal replacement or snack comprising the composition. In an aspect, the meal replacement or snack is an nutrition bar or an energy bite.

Compositions according to the present disclosure may also comprise other ingredients that can modify the chemical, physical or processing characteristics of the products or serve as pharmaceutical or additional nutritional components when they are used by certain target populations or cohorts.

Optional ingredients known or otherwise adapted for use in other food products may also be used in the compositions in accordance with the present disclosure, on condition that these optional ingredients are safe and efficient for consumption, administration, and are compatible with the other essential ingredients of the composition.

Non-limiting examples of such optional ingredients comprise buffers, pharmaceutical active agents, additional nutrients, dyes, flavorings, thickeners and stabilizers, etc.

In some embodiments, the composition is in powder or liquid form.

The composition in powder or liquid form may also comprise one or more masking agents to reduce, for example, the bitter tastes in reconstituted powders. Suitable masking agents comprise natural and artificial sweeteners, such as and not limited to, stevia, honey, saccharine, sucralose and aspartame, sources of sodium, such as sodium chloride, and hydrocolloids such as guar gum, xanthan gum, carrageenan, and combinations thereof.

In other embodiments, the composition comprises a flavorant or flavoring agent, including and not limited to, chocolate, cocoa, carob powder, cinnamon, mocha or vanilla, or a combination thereof.

Compositions of the present disclosure may be reconstituted with water or another aqueous liquid, including and not limited to, coconut water, dairy milk, nut milk, coffee, fruit or vegetable juice. In other embodiments, the present disclosure relates to the application of these compositions as beverages, for non-limiting example, ready-to-drink beverages, or nutritional liquids, by means of powder mixes to be reconstituted, with water or another aqueous liquid. For non-limiting example, the composition is reconstituted as ready-to-drink beverages, in which low viscosity of the beverage is sought.

Compositions of the present disclosure in powder form is prepared by, for non-limiting example, mixing various powders.

### Delivery, Administration, and Devices

Compositions described herein can be administered (e.g., self-administered) by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), ophthalmic, mucosal, nasal, bucal, sublingual; by intratracheal instillation, bronchial instillation, and/or inhalation; and/or as an oral spray, nasal spray, and/or aerosol. In general, the most appropriate route of administration will depend upon a variety of factors, such as the nature of the agent (e.g., its stability in the environment of the gastrointestinal tract), and/or the condition of the subject (e.g., whether the subject is able to tolerate oral administration). In some embodiments, a composition is formulated for oral administration. In some embodiments, the mode of administration is selected based on a subject's symptoms; for example, inhalation-based delivery may be preferable in cases of nausea, where oral administration is less tolerable, whereas transdermal or sublingual routes may be utilized for subjects requiring rapid onset without gastrointestinal involvement.

One or more products described herein may be administered to a subject at a given time (e.g., the times of use may vary from once a day, twice a day, three times a day, in the morning, at mealtime, at bedtime, or use as needed, specified in a treatment plan).

In certain instances, it may be advantageous to administer a first cannabinoid or a second cannabinoid in combination with one or more additional therapeutic agent(s). For example, it may be advantageous to administer a first cannabinoid or a second cannabinoid in combination with one or more additional therapeutic agents, e.g., independently selected from an anti-cancer agent (e.g., chemotherapeutic agent), immunotherapy (e.g., an immune checkpoint inhibitor), anti-allergic agent, anti-emetic, pain reliever, immunomodulator and cytoprotective agent. The agents may be administered simultaneously, sequentially, in the same composition, in separate compositions. In some embodiments, a cannabinoid is administered with melatonin.

The present disclosure relates to delivery systems comprising a composition of the present disclosure. Example delivery systems include, but are not limited to, gummies, tablets, capsules, soft gels, chewables, lozenges, powders, oral dissolvable films, transdermal patches, sublingual strips, beverages, sprays, suppositories, creams, and ointments. In some embodiments, the delivery system comprises a gummy, a tablet or a capsule. In some embodiments, the delivery system comprises a capsule. In some embodiments, the capsule is a micro-pelletized capsule, soft gel capsule, hard shell capsule, liquid-filled capsule, enteric-coated capsule, sustained-release capsule, or a combination thereof. In some embodiments, the capsule is a micro-pelletized capsule. As used herein, the term “micro-pelletized” refers to pellets having undergone a process in which a cured solution is spray-coated onto a core (e.g., an inert core). The solution that cures around the core may contain an active ingredient such as one or more cannabinoid (e.g., THC, CBN) or agent such as melatonin or protein, and a second coat, which does not contain an active ingredient, may be applied to achieve a desired thickness. Multiple layers (at least one layer with active ingredient) created in this process may result in varying dissolution rates, allowing for extended-release properties. In some embodiments, the capsule contains pellets having diameter of from about 0.84 mm to about 0.25 mm.

The present disclosure also relates to devices comprising one or more of: a delivery system (e.g., dosage forms) comprising a composition of the present disclosure; a controlled dosing mechanism to regulate the amount of the composition delivered to a subject; and at least one safety feature to prevent misuse and ensure compliance of the subject with recommended dosing protocols.

Examples of delivery systems for a composition of the present disclosure include solid dosage forms for oral administration such as capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (c) solution retarding agents such as paraffin, (f) absorption accelerators such as quaternary ammonium compounds, (g) wetting agents, such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes. In some embodiments, the coatings can contain multiple (2, 3 or more) cannabinoids and/or agents, e.g., melatonin. In some embodiments, cannabinoids and/or agents can be included separately on additional coatings. For non-limiting example, a sleep product could include THC, CBN and melatonin in each of one, two or three layers.

Dosage forms for topical and/or transdermal administration of a product or compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

In some embodiments, a delivery system comprises a transdermal patch or a controlled-release capsule.

In other embodiments, a delivery system comprises an inhaler, a vaporization system, or a combination thereof, wherein the vaporization system is configured to heat the composition to a temperature suitable for vaporization without combustion.

Liquid dosage forms are also contemplated herein, for example, for oral and parenteral administration, include pharmaceutically acceptable emulsions, microemulsions, solutions, suspensions, syrups and elixirs. In some embodiments the dosage form comprises a nanoemulsion. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the active ingredient is mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent.

Compositions for rectal or vaginal administration may be delivered using delivery systems such as suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Compositions described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization delivery system. Such formulations may further comprise one or more additional ingredients including, but not limited to, a flavoring agent such as saccharin sodium, a volatile oil, a buffering agent, a surface-active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Compositions described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

Although the descriptions of compositions provided herein are principally directed to compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compositions described herein are typically formulated in dosage unit form for ease of administration and uniformity of dosage. It will be understood, however, that the total daily usage of such forms will be decided by a physician within the scope of sound medical judgment. The specific therapeutically effective dose level for any particular subject or organism will depend upon a variety of factors including, for example, the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a therapeutic agent in a composition required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses may include different or substantially the same amounts of a therapeutic agent, such as a compound described herein. In certain embodiments, when multiple doses are administered to a subject or applied to a tissue or cell, the frequency of administering the multiple doses to the subject or applying the multiple doses to the tissue or cell is three doses per day, two doses per day (e.g. BID), one dose per day (e.g., QD), one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

In certain embodiments, a dose (e.g., a single dose, or any dose of multiple doses, a unit dosage form) includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound (e.g., a cannabinoid, an agent) described herein. In certain embodiments, a dose includes independently between 1 mg and 3 mg, inclusive, of a compound described herein. In certain embodiments, a dose includes independently between 3 mg and 10 mg, inclusive, of a compound described herein. In certain embodiments, a dose includes independently between 10 mg and 30 mg, inclusive, of a compound described herein. In certain embodiments, a dose includes independently between 30 mg and 100 mg, inclusive, of a compound described herein. In certain embodiments, a dose includes independently between 10 mg and 250 mg, inclusive, of a compound described herein. In certain embodiments, a dose includes independently between 10 mg and 100 mg (e.g., about 45 mg, about 75 mg, about 90 mg), inclusive, of a compound described herein.

For example, the compositions escribed herein can be in a unit dosage form containing from about 1 to about 1000 mg of active ingredient(s) (e.g., for a subject of from about 50 to about 70 kg), or from about 1 to about 500 mg, from about 1 to about 250 mg, from about 1 to about 150 mg, from about 0.5 to about 100 mg, or from about 1 to about 50 mg of active ingredient(s) (e.g., for a subject of from about 50 to about 70 kg). The therapeutically effective dosage of a compound or composition is dependent on the species of the subject, the body weight, age and individual condition of the subject, and the disease, disorder or condition or the severity thereof being treated. A physician, clinician or veterinarian of ordinary skill can readily determine the therapeutically effective amount of each of the active ingredients necessary to prevent or treat the progress of the disease, disorder or condition.

Compositions can also be formulated so as to deliver a particular dose to a subject. A dose may range, depending on the route of administration, among other things, between about 0.1 mg/kg to about 500 mg/kg subject mass, or between about 1 mg/kg to about 100 mg/kg subject mass. In some embodiments, the dosage is expected to be in the range of 1 mg/Kg subject mass and 150 mg/Kg subject mass, for example, at least about 1 mg/Kg, at least about 10 mg/Kg, at least about 20 mg/Kg, at least about 30 mg/Kg, at least about 40 mg/Kg, at least about 50 mg/Kg, at least about 60 mg/Kg, at least about 70 mg/Kg, at least about 80 mg/Kg, at least about 90 mg/Kg, at least about 100 mg/Kg, at least about 110 mg/Kg, at least about 120 mg/Kg, at least about 130 mg/Kg, at least about 140 mg/Kg, or about 150 mg/Kg.

In some embodiments, dose ranges described herein provide guidance for the administration of provided pharmaceutical compositions to an adult. The amount to be administered to, for example, a child or an adolescent, can be determined by a medical practitioner or person skilled in the art and can be lower or the same as that administered to an adult.

Devices of the present disclosure may further comprise a user interface integrated with a digital platform to provide real-time feedback and guidance to the subject on usage, dosing, and treatment adherence; and wireless connectivity for remote monitoring to track treatment progress and therapeutic outcomes for the subject. In some embodiments, the device comprises an Internet of Things (IoT) gadget. The IoT (internet of things) connectivity allows remote control and monitoring.

Digital platforms (e.g., telehealth platforms) for providing real-time feedback and guidance to the subject are disclosed in U.S. patent application Ser. No. 17/888,260, which is incorporated herein by reference in its entirety.

One or more components of the devices of the present disclosure, such as the digital platform, is installed on one or more underlying computing platforms, including on-premise platforms, cloud computing platforms and/or a combination thereof, such as hybrid cloud platforms. An on-premise platform is a computing platform that may be installed and operated on the premises of an entity such as a customer of the on-premise platform. A cloud computing platform may span wide geographic locations, including countries and continents. The service and/or application components (e.g., tenant infrastructure or tenancy) of the cloud computing platform may include nodes (e.g., computing devices, processing units, or blades in a server rack) that are allocated to run one or more portions of a tenant's services and applications. When more than one service or application is being supported by the nodes, the nodes may be partitioned into virtual machines or physical machines.

Communication between the respective components of the devices of the present disclosure is possible using an API (Application Programming Interface) implemented to send and/or receive information between, for example, the controlled dosing mechanism and digital platform Configuration of such API's employ technology and techniques that are common or known in the art and are within the purview of one skilled in the art given the disclosure herein.

Furthermore, embodiments may be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks may be stored in a machine-readable medium such as a computer-readable storage medium. A code segment or machine executable instruction may represent a procedure, a function, a subprogram, a program, a routine, a subroutine, a module, a software package, a script, a class, or any combination of instructions, data structures, and/or program statements. A code segment may be coupled to another code segment or a hardware circuit by passing and/or receiving information, data, arguments, parameters, and/or memory contents. Information, arguments, parameters, data, etc. may be passed, forwarded, or transmitted via any suitable means including memory sharing, message passing, token passing, network transmission, etc.

Computer-readable medium includes both non-transitory computer storage medium and communication medium including any medium that facilitates transfer of a computer program from one place to another. A non-transitory computer-readable storage medium includes any available medium that can be accessed by a general purpose or special purpose computer. By way of example, and not limiting, non-transitory computer-readable storage medium can comprise Random Access Memory (RAM), read Only Memory (ROM), electrically Erasable Programmable Read Only Memory (EEPROM), flash memory, compact Disc (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital Subscriber Line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes CD, laser disc, optical disc, digital Versatile Disc (DVD), floppy disk and blu-ray disc where disks usually reproduce data magnetically, while discs reproduce data optically with lasers. Combinations of the above are also included within the scope of computer-readable medium.

### Kits

Also encompassed by the disclosure are kits (e.g., pharmaceutical packs). The kits provided may comprise a composition (e.g., a pharmaceutical composition) of the present disclosure, and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound contained in the kit. In some embodiments, compositions described herein provided in the first container and the second container are combined to form a unit dosage form.

Thus, in one aspect, provided are kits including a first container comprising host protein inhibitors of the disclosure, or a pharmaceutical composition thereof. In certain embodiments, the kits are useful in one or more of the methods described herein, for example, for treating a disease (e.g., a proliferative disease) in a subject in need thereof. In certain embodiments, the kits are useful for preventing a disease in a subject in need thereof. In certain embodiments, the kits are useful for reducing the risk of developing a disease in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information.

A composition (or formulation) for application may be packaged in a variety of ways depending upon the method used for administering the composition. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

In some embodiments, the concentration of one or more cannabinoids or agent (e.g., therapeutic agent) provided in a composition is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of one or more cannabinoids or agent (e.g., therapeutic agent) in a composition is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of one or more cannabinoids or agent (e.g., therapeutic agent) provided in a composition is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12%, about 1% to about 10% w/w, w/v or v/v.

In some embodiments, the concentration of one or more cannabinoids or agent (e.g., therapeutic agent) provided in a composition is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v.

### Methods and Functional Benefits

The disclosure provides, among other things, methods of improving an outcome of a subject. In some embodiments, the methods comprise: administering a first composition comprising a first cannabinoid to the subject. In some embodiments, improving an outcome of a subject comprises one or more of: (i) reducing a condition of the subject, (ii) improving mobility of the subject and reducing difficulty of performing general activities and mobility by the subject, (iii) reducing difficulty of performing daily living activities by the subject, or (iv) reducing difficulty of performing activities related to work, social, or leisure time by the subject.

In some embodiments, improving an outcome of a subject comprises or consists of reducing a condition of the subject. In some embodiments, improving an outcome of a subject comprises or consists of improving mobility of the subject and reducing difficulty of performing general activities and mobility by the subject. In some embodiments, improving an outcome of a subject comprises or consists of reducing difficulty of performing daily living activities by the subject. In some embodiments, improving an outcome of a subject comprises or consists of reducing difficulty of performing activities related to work, social, or leisure time by the subject. In some embodiments, the first composition comprising the first cannabinoid is administered to the subject based on a treatment plan. In some embodiments, the treatment plan is: based on subject's data; and provided by a digital platform comprising a model-based treatment plan generator. In some embodiments, the first cannabinoid is tetrahydrocannabinol, tetrahydrocannabivarin, or a combination thereof.

In some embodiments, the methods comprise: providing a treatment plan based on subject's data; and administering a composition comprising a first cannabinoid to the subject based on the treatment plan.

In some embodiments, methods of improving an outcome of a subject comprise: administering a composition comprising a cannabinoid to the subject based on a treatment plan that is based on the subject's data, the cannabinoid intended to cause an improved outcome of the subject, wherein the outcome comprises one or more of: (i) reducing a condition of the subject, (ii) improving mobility of the subject and reducing difficulty of performing general activities by the subject, (iii) reducing difficulty of performing daily living activities by the subject, or (iv) reducing difficulty of performing activities related to work, social, or leisure time by the subject.

Subject's data includes information provided by the subject that is used to personalize the treatment plan. Subject's data may include medical history, symptoms, current medications, allergies, lifestyle factors, treatment goals, mental health status, emotional well-being, risk factors, etc. For example, subject's data may comprise one or more of: a relaxation level of the subject, an energy level of the subject, a mental level of the subject, a euphoria level of the subject, a mood level of the subject, or whether the subject experienced undesired side effects from existing medications.

In some embodiments, an outcome may be quantified using tools such as standardized questionnaires or scales which allows a subject (e.g., a patient) to self-report measures of a subject's health status or behavior, thereby coming directly from the subject without requiring interpretation from anyone else. For example, these tools may capture subject-relevant lifestyle physical activities and related features that may not be identified by other assessments. For example, a subject may rate the difficulty of performing general or specific activities (e.g., dressing, bathing, walking, cooking) on a scale, such as “0 to 4” scale (0=no difficulty, 4=unable to perform), “0 to 10” scale (0=no difficulty, 10=extreme difficulty), etc. More examples of such questionnaires are disclosed in Williams, K., Frei, A., Vetsch, A., Dobbels, F., Puhan, M. A. and Rüdell, K., 2012. Patient-reported physical activity questionnaires: a systematic review of content and format. Health and quality of life outcomes, 10, pp. 1-18, the contents of which are incorporated herein by reference in their entirety.

In some embodiments, the treatment goals of a subject comprise subject-specified goals directed toward improving an outcome (e.g., a patient-identified outcome, etc.). For example, a treatment goal of a subject may include the ability to walk independently for at least 10 minutes without rest, which reflects a subject-specified goal directed towards improving a patient-identified outcome related to mobility. A subject may record their progress towards their treatment goals and rate their achievement over time on a scale (e.g., 5-point scale: −2=much less than expected outcome, 0=expected level, and +2=much more than expected). As another example, a treatment goal of a subject may include the ability to attend weekly social gatherings without experiencing overwhelming anxiety, which reflects a subject-specified goal directed towards improving an outcome related to leisure activities.

In some embodiments, providing a treatment plan based on subject's data comprises utilizing a digital platform comprising a model-based (e.g., Bayesian model-based) treatment plan generator to generate the treatment plan based on subject's data. In some embodiments, subject's data comprises: health profile of the subject comprising one or more of: severity of the condition of the subject, the mobility of the subject and the difficulty of performing general activities by the subject, the difficulty of performing daily living activities by the subject, or the difficulty of performing activities related to work, social, or leisure time by the subject; and treatment goals of the subject. In some embodiments, subject's data comprises: health profile of the subject comprising severity of pain, sleep disturbance, anxiety, or gastrointestinal nausea and vomiting of the subject; or treatment goals of the subject.

Example benefits of using a Bayesian model are disclosed in U.S. patent application Ser. No. 17/888,260, the contents of which are incorporated herein by reference in its entirety. For example, a Bayesian model allows for an incomplete dataset, e.g., the digital platform may be missing one or more datapoints from a user (e.g., a user may miss one or more sessions, fail to provide a detailed health profile to the platform), but the Bayesian model can use assume priors to put a reasonable guess into what that datapoint would be.

In some embodiments, the digital platform may include a model (e.g., an NLP model) which receives subject's data (e.g., open voice to text and text entry inputs). The model may process the subject (input) data into probabilistic multiple choice outputs that can be used by other models in the digital platform, such as a regimen recommendation model (e.g., a Bayesian model). In some embodiments, the model-based treatment plan generator is the regimen recommendation model. An example of an open voice input may include a question that asks the user to talk about their pain diagnosis.

The digital platform may train the regimen recommendation model using research data associated with cannabis, clinical trials associated with cannabis, and/or data indicative of physician expertise and experience associated with cannabis. In some embodiments, the digital platform may be configured to further train (e.g., tune) the regimen recommendation model by matching physician recommendations to the model output. The digital platform may continually re-train (e.g., update) the recommendation model using a dataset generated from one or more of the users of the platform, their user (i.e., subject) profile data, all their feedback inputs, the careplan attributes for recommended products by phase, and/or their progress in well-being and activity improvement metrics. In some embodiments, the digital platform may estimate the one or more probabilities in each node of the regimen recommendation model based on trial results and physician knowledge and experiences to represent the uncertainty throughout the model. In some embodiments, the digital platform may also train the regimen recommendation model using information from one or more user profiles, where the information includes user responses that are associated with different dayparts (e.g., time segments).

In some embodiments, the regimen recommendation model may be configured to make recommendations (e.g., which product to use, and when to use the product) based on a subject's feelings (or treatment goals) that are associated with different dayparts, as indicated in the subject profile for the subject.

In some embodiments, the regimen recommendation model may be configured to make recommendations based on cannabis tolerance (e.g., body type factors and/or a subject's cannabis experience), plan intensity; general CBD: THC type (e.g., determined by the subject's diagnosis and tolerance), condition (e.g., pain, anxiety, depression, sleeplessness, autism spectrum disorders (ASD), etc.), how the subject wants to feel by daypart (e.g., maintain mental clarity, relax, energetic, euphoric, etc.), which times of day the subject has worse pain (e.g., morning, afternoon, evening, nighttime), and/or type of day (e.g., workday/school day, non-workday/home day, etc.)

In some embodiments, the digital platform may be configured to determine the plan intensity by the severity of the subject's pain, cannabis tolerance, and/or impact of other medication the subject is taking. In some embodiments, the digital platform may be configured to determine a general CBD: THC type by the subject's diagnosis and tolerance.

The regimen recommendation model may be configured to make recommendations that include a more frequent/higher dosing throughout the day, which may benefit those subjects that have a higher pain and/or more intense symptoms. For example, a subject with high pain in the morning on a workday may have a CBD-dominant product for mental clarity, with a micro-dosed fast-acting THC-dominant product for a pain-relieving boost that is easily titratable to adjust as needed for maintaining mental clarity.

The regimen recommendation model may be configured to make recommendations based on a subject's feeling goal for a particular daypart, as indicated in the subject profile for the subject. For example, a subject may want to use a full THC-dominant product in the morning on a non-workday when mental clarity is not a priority, and given that this is morning, maybe they also want energy. In that case, the regimen recommendation model may recommend a product that will be a strain with terpenes or other herbal ingredients that promote energy. As another example, the subject's data may indicate that a subject gets sleepy with a particular strain that is energizing for other subjects. In this embodiment, regimen recommendation model might recommend an alternate strain (an alternate product) and further learn how this particular subject reacts to strains/terpenes/other cannabinoids/other herbal supplements.

In some embodiments, the generated treatment plan is displayed on a user device (e.g., computer, cellphone). The treatment plan may include recommended products, dosages, times of day for use, instructions, and explanations on why the products were selected.

The treatment plan may be updated based on specific needs, preferences, and/or progress of the subject. For example, a subject may provide feedback or updated treatment goals to the platform via a user interface (e.g., a subject may use a cellphone or computer coupled to a digital platform to access a portal and record their progress toward treatment goals). For example, a subject may log daily pain levels, note improvements in mobility, or report the ability to complete specific physical activities. The digital platform may analyze the data and generate an updated treatment plan tailored to the subject's current needs or goals.

In some embodiments, methods of the present disclosure further comprises: providing a maintained or an updated treatment plan based on updated subject's data. For example, based on updated subject's data, a digital platform may provide to the subject the same treatment plan as the existing plan, or modify the existing treatment plan. In some embodiments, updated subject's data comprises one or more of: feedback from the subject, or treatment adherence data. In some embodiments, an updated treatment plan comprises one or more of: adjusted medication dosages, modifications to regimens, or recommendations for lifestyle changes.

In some embodiments, reducing a condition of a subject comprises reducing one or more symptoms associated with a disease or a therapy. In some embodiments, the disease is cancer. In some embodiments, the therapy is chemotherapy. In some embodiments, the disease is cancer, and the therapy is chemotherapy.

In some embodiments, a condition of a subject comprises chronic pain, inflammation, cancer symptoms, sleep disturbances, anxiety, or a combination thereof.

In some embodiments disclosed herein are methods of reducing pain, sleep disturbance, anxiety, or gastrointestinal nausea and vomiting in a subject, the method comprising: administering a first composition comprising a first cannabinoid to the subject.

In some embodiments, a method of reducing pain in a subject comprises administering a first composition comprising a first cannabinoid to the subject.

In some embodiments, a method of reducing sleep disturbance in a subject comprises administering a first composition comprising a first cannabinoid to the subject.

In some embodiments, a method of reducing anxiety in a subject comprises administering a first composition comprising a first cannabinoid to the subject.

In some embodiments, a method of reducing gastrointestinal nausea and vomiting in a subject comprises administering a first composition comprising a first cannabinoid to the subject.

In some embodiments, the first composition comprising the first cannabinoid is administered to the subject based on a treatment plan. In some embodiments, the treatment plan is: based on subject's data; and provided by a digital platform comprising a model-based treatment plan generator.

In some embodiments disclosed herein are methods of reducing pain, sleep disturbance, anxiety, or gastrointestinal nausea and vomiting in a subject, the methods comprising: providing a treatment plan based on subject's data; and administering a composition comprising a first cannabinoid to the subject based on the treatment plan.

In some embodiments disclosed herein are methods of reducing pain, sleep disturbance, anxiety, or gastrointestinal nausea and vomiting in a subject, the methods comprising: administering a composition comprising a cannabinoid to the subject based on a treatment plan that is based on the subject's data, the cannabinoid intended to cause reduced pain, sleep disturbance, anxiety, or gastrointestinal nausea and vomiting in the subject.

Methods of the present disclosure may further comprise: administering a second composition comprising a first cannabinoid to a subject. In some embodiments, the second composition comprising the first cannabinoid is administered to the subject based on an updated treatment plan. In some embodiments, the updated treatment plan is based on updated subject's data and provided by a digital platform comprising a model-based treatment plan generator. In some embodiments, the updated subject's data comprises one or more of: feedback from the subject, or treatment adherence data.

In some embodiments, a composition is a first composition and a cannabinoid is a first cannabinoid, and wherein a method of the present disclosure further comprises: administering a second composition comprising the first cannabinoid to a subject.

In some embodiments, the first composition further comprises a second cannabinoid. In some embodiments, the second composition further comprises a second cannabinoid. In some embodiments, the second cannabinoid is cannabigerol, cannabichromene, cannabinol, delta-8-tetrahydrocannabinol, tetrahydrocannabinolic acid, cannabidiolic acid, or a combination thereof. A second composition may be identical to a first composition, or different from a first composition.

Also disclosed herein, in an embodiment, is a computer-implemented method for providing a treatment plan for improving an outcome of a subject, the method comprising: enabling the subject to enter data, representative of a condition of the subject for which the subject is being provided with a treatment plan, to a processing module; by the processing module, determining a treatment plan based on a combination of the data and a composition comprising a cannabinoid, the module providing the treatment plan; and enabling the subject to enter updated data, the processing module, in turn, determining an updated treatment plan based on a combination of the updated data and the composition comprising the cannabinoid, the processing module providing notice to the subject to maintain or update the composition or the treatment plan, wherein the treatment plan is intended to cause an improved outcome of the subject, wherein the outcome comprises one or more of: (i) reducing a condition of the subject, (ii) improving mobility of the subject and reducing difficulty of performing general activities by the subject, (iii) reducing difficulty of performing daily living activities by the subject, or (iv) reducing difficulty of performing activities related to work, social, or leisure time by the subject.

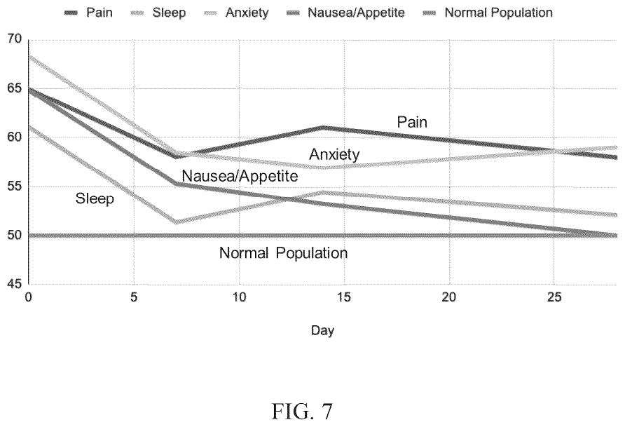

As an example, a subject is provided with a treatment plan at Day 0 based on initial subject's data. A computer-implemented method may enable the subject to enter updated data and the processing module in turn determines an updated treatment plan for the subject at Day 5, or the treatment plan at Day 5 may be identical to the treatment plan at Day 0. Over the course of treatment, the subject may enter updated data at various time points and the treatment plan may be maintained or updated, with the goal of improving an outcome (e.g., reducing pain, reducing nausea, etc.) over the course of treatment. An example of improved outcomes is illustrated in FIG. 7.

The disclosure is also directed to methods for achieving one or more functional benefits in a subject in need thereof by consuming the compositions described herein. The presence of cannabinoids and their use levels in the compositions harnesses the functional benefits. Many disease states may be prevented or improved in a subject by routine consumption of the compositions of the disclosure.

Example functional benefits include, but are not limited to, reducing one or more of: chronic pain, inflammation, cancer symptoms, sleep disturbances and anxiety. Other examples include sustained steady energy, improving vascular health, enhancing immune system, improving cognitive function, improving metabolism, or a combination thereof. The composition may improve, or alleviate a condition or disease, or a plurality of conditions or diseases, including, but not limited to, proliferative diseases, neurological conditions or diseases, inflammatory conditions or diseases, and metabolic conditions or diseases.

In some embodiments provided herein are methods for reducing or managing symptoms associated with cancer or cancer-related treatments (e.g., chemotherapy) in a subject (e.g., a subject in need thereof), the methods comprising administering to the subject a therapeutically effective amount of a composition.

A wide variety of cancers, including solid tumors, leukemias, lymphomas, and myelomas are amenable to the methods disclosed and contemplated herein. In some embodiments, the cancer is a solid tumor cancer. In some embodiments, the cancer comprises a solid tumor (e.g., a colorectal, breast, prostate, lung, pancreatic, renal or ovarian tumor). Accordingly, in some embodiments, the cancer is a solid tumor cancer. In some embodiments, the cancer is selected from one or more of a cancer of the pulmonary system, a brain cancer, a cancer of the gastrointestinal tract, a skin cancer, a genitourinary cancer, head and neck cancer, a sarcoma, a carcinoma, and a neuroendocrine cancer. In various embodiments, the solid tumor cancer is breast cancer, bladder cancer, endometrial cancer, esophageal cancer, liver cancer, pancreatic cancer, lung cancer, cervical cancer, colon cancer, colorectal cancer, gastric cancer, kidney cancer, ovarian cancer, prostate cancer, testicular cancer, uterine cancer, a viral-induced cancer, melanoma or sarcoma. In some embodiments, the cancer is cervical cancer. In some embodiments, the cancer is bladder cancer. In some embodiments, the cancer is lung cancer (e.g., non-small cell lung cancer). In other embodiments, the cancer is liver cancer. In some embodiments, the cancer is a sarcoma, bladder cancer or renal cancer. In some embodiments, the cancer is gastric cancer. In some embodiments, the cancer is breast cancer. In some embodiments, the cancer is ovarian cancer. In some embodiments, the cancer is pancreatic cancer. In some embodiments, the cancer is mesothelioma. In some embodiments, the cancer is prostate cancer (e.g., castration-resistant prostate cancer, castration-sensitive prostate cancer). In other embodiments, the cancer is bladder cancer, pancreatic cancer, colorectal cancer, glioblastoma, kidney cancer, non-small cell lung carcinoma, prostate cancer, sarcoma, skin cancer, thyroid cancer, testicular cancer or vulvar cancer. In some embodiments, the cancer is endometrial cancer, pancreatic cancer, testicular cancer, renal cancer, melanoma, colorectal cancer, thyroid cancer, bladder cancer, pancreatic cancer, vulvar cancer, sarcoma, prostate cancer, lung cancer or anal cancer. In some embodiments, the cancer is a sarcoma. In some embodiments, the cancer is a renal cell carcinoma. In particular embodiments, the cancer is ovarian granulosa cell tumor (e.g., adult granulosa cell tumor (AGCT), pediatric granulosa cell tumor).

In some embodiments, the cancer is a non-solid tumor cancer. In some embodiments, the cancer is a hematologic cancer. Hematologic cancers that can be treated according to the methods described herein include leukemias (e.g., acute leukemias, chronic leukemias), lymphomas (e.g., B-cell lymphoma, T-cell lymphoma) and multiple myeloma. In some embodiments, the hematologic cancer is selected from multiple myeloma, myelodysplastic syndrome (MDS), acute myeloid leukemia (AML), acute lymphoblastic leukemia (ALL), acute lymphocytic leukemia, lymphocytic lymphoma, mycosis fungoides, chronic lymphogenous leukemia, chronic lymphocytic leukemia (CLL), mantle cell lymphoma, diffuse large B-cell lymphoma, follicular lymphoma, Hodgkin's lymphoma, non-Hodgkin's lymphoma or myelofibrosis.

Example symptoms or effects of cancer, cancer-related treatments (e.g., chemotherapy), or any other disease or condition, that are addressed (e.g., treated, managed, reduced) according to the products and methods described herein include, but are not limited to, nausca, spasticity, pain, insomnia, sexual health issues or discomfort with sex, fatigue, anxiety, depression, cognitive impairment, loss of appetite, cachexia, neuropathic pain, gastrointestinal disturbances, mucositis, arthritic pain, anxiety, sleeplessness, and hot flashes.

In some embodiments, the composition alleviates a condition or disease, or reduces the condition or disease in severity, duration or frequency of occurrence, or alleviates at least one symptom associated with the condition or disease, or reduces in severity, duration, or frequency of occurrence. In some embodiments, the condition or disease is an inflammatory or metabolic condition or disease.

Methods of the present disclosure may further comprise utilizing a digital platform comprising a model-based treatment plan generator prior to administering a composition of the present disclosure to a subject in need thereof, wherein the utilizing comprises analyzing subject's data and generating personalized treatment plans tailored to characteristics and treatment goals of the subject. In some embodiments, the analyzing comprises identifying health profiles, symptom severity, and treatment responses of the subject.

Methods of the present disclosure may further comprise monitoring subject-reported outcomes and treatment adherence data after administering a composition of the present disclosure to a subject in need thereof. In some embodiments, the monitoring comprises one or more of: providing the subject with access to a digital platform for real-time feedback, symptom tracking, and communication with healthcare providers; and identifying trends, patterns, and interventions for enhancing therapeutic efficacy and subject satisfaction. Predictive analytics and models may be used to identify trends, patterns, and interventions.

The present disclosure also relates to methods of forming a cannabinoid-based product for a subject in need thereof, comprising one or more of: identifying symptoms, concerns, treatment side effects, or a combination thereof experienced by the subject; providing a composition of the present disclosure based on the symptoms, concerns, treatment side effects, or the combination thereof; and incorporating the composition into a non-combustible form factor to ensure safe and consistent delivery of the composition, thereby forming the cannabinoid-based product (e.g., a micro-pelletized capsule) for the subject. Methods of the present disclosure may further comprise integrating a digital platform for personalized dosing regimens, remote monitoring of therapeutic outcomes for the subject, and adjusting the regimens in real-time based on responses from the subject to optimize the therapeutic outcomes. In some embodiments, one or more of steps of methods of the present disclosure may be performed by a digital platform disclosed herein. For example, providing a composition of the present disclosure based on a subject's symptoms, concerns, treatment side effects, or the combination thereof may be performed by a digital platform disclosed herein.

In some embodiments, an agent comprises proteins, amino acids, fibers, vitamins, minerals, or a combination thereof.

Also disclosed herein are methods of delivering a composition to a subject, the methods comprising administering to the subject a composition of the present disclosure via a device or a delivery system of the present disclosure.

### Definitions

It is to be understood that the terminology used herein is for describing particular embodiments only and is not intended to be limiting. Unless defined otherwise, all technical and scientific terms used herein have the same meaning as commonly understood by one of ordinary skill in the art to which the disclosure pertains.

As used herein, “model” includes, and is not limited to, classification models, time series models, neural network models, linear regression models, logistic regression models, decision trees, support vector machines, Naive Bayes networks, k-nearest neighbor (KNN) models, k-means models, random forest models, association rule learning models, inductive logic programming models, reinforcement learning models, feature learning models, similarity learning models, sparse dictionary learning models, genetic algorithm models, rule-based machine learning models, learning classifier system models, or any combination thereof. In some embodiments, a model comprises a Bayesian model.

Compounds (e.g., cannabinoids, agents) described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). Additionally, encompassed are compounds as individual isomers substantially free of other isomers, and alternatively, as mixtures of various isomers.

As used herein, the term “isomers” refers to different compounds that have the same molecular formula but differ in arrangement and configuration of the atoms.

“Enantiomers” are a pair of stereoisomers that are non-superimposable mirror images of each other. A 1:1 mixture of a pair of enantiomers is a “racemic” mixture. “Racemate” or “racemic” is used to designate a racemic mixture where appropriate. When designating the stereochemistry for the compounds of the present disclosure, a single stereoisomer with known relative and absolute configuration of the two chiral centers is designated using the conventional RS system (e.g., (1S,2S)); a single stereoisomer with known relative configuration but unknown absolute configuration is designated with stars (e.g., (1R*,2R*)); and a racemate with two letters (e.g., (1RS,2RS) as a racemic mixture of (1R,2R) and (1S,2S); (1RS,2SR) as a racemic mixture of (1R,2S) and (1S,2R)). “Diastereoisomers” are stereoisomers that have at least two asymmetric atoms, but which are not mirror-images of each other. The absolute stereochemistry is specified according to the Cahn-Ingold-Prelog R-S system. When a compound is a pure enantiomer, the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Alternatively, the resolved compounds can be defined by the respective retention times for the corresponding enantiomers/diastereomers via chiral HPLC.

Geometric isomers may occur when a compound contains a double bond or some other feature that gives the molecule a certain amount of structural rigidity. If the compound contains a double bond, the double bond may be E- or Z-configuration. If the compound contains a disubstituted cycloalkyl, the cycloalkyl substituent may have a cis- or trans-configuration.

Conformational isomers (or conformers) are isomers that can differ by rotations about one or more bonds. Rotamers are conformers that differ by rotation about only a single bond.

The term “atropisomer,” as used herein, refers to a structural isomer based on axial or planar chirality resulting from restricted rotation in the molecule.

Optically active (R)- and(S)-isomers may be prepared using chiral synthons or chiral reagents, or resolved using conventional techniques (e.g., separated on chiral SFC or HPLC chromatography columns, such as CHIRALPAK® and CHIRALCEL® columns available from DAICEL Corp. or other equivalent columns, using the appropriate solvent or mixture of solvents to achieve suitable separation).

Compounds disclosed herein can be isolated in optically active or racemic forms. Optically active forms may be prepared by resolution of racemic forms or by synthesis from optically active starting materials. All processes used to prepare compounds and intermediates disclosed herein are considered to be part of the present disclosure. When enantiomeric or diastereomeric products are prepared, they may be separated by conventional methods, for example, by chromatography or fractional crystallization.

As used herein, the term “salt” refers to any and all salt forms that compounds disclosed herein can be prepared as, and encompasses pharmaceutically acceptable salts. Pharmaceutically acceptable salts are preferred. However, other salts may be useful, e.g., in isolation or purification steps which may be employed during preparation, and thus, are contemplated to be within the scope of the present disclosure. In general, salts of a compound described herein will be those that provide a composition suitable for administration to a human or animal subject via any suitable route of administration of a pharmaceutical composition.

The term “pharmaceutical composition” as used herein, denotes a composition in which at least one therapeutic agent retains, or partially retains, its intended biological activity or functional form, and in which only pharmaceutically acceptable components are included.

The phrase “pharmaceutically acceptable” means that the substance or composition the phrase modifies must be, within the scope of sound medical judgment, suitable for use in contact with the tissues of humans and lower animals without undue toxicity, irritation, allergic response and the like, and are commensurate with a reasonable benefit/risk ratio. If a substance is part of a composition or formulation, the substance must also be compatible chemically and/or toxicologically with the other ingredients in the composition or formulation.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference, and for example, lists of suitable salts are found in Allen, L. V., Jr., ed., Remington: The Science and Practice of Pharmacy, 22nd Edition, Pharmaceutical Press, London, UK (2012). Pharmaceutically acceptable salts of the compounds described herein include those derived from suitable inorganic and organic acids and inorganic and organic bases.

Pharmaceutically acceptable acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art, such as ion-exchange. Other pharmaceutically acceptable acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

Pharmaceutically acceptable base addition salts are formed from inorganic and organic bases. Salts derived from appropriate bases include alkali metal, alkaline earth metal, ammonium, and N⁺ (C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations (e.g., primary, secondary, tertiary, quaternary amine cations), for example, formed using counterions such as halide, hydroxide, carboxylate, sulfate, phosphate, nitrate, lower alkyl sulfonate, and aryl sulfonate. Examples of organic amines from which base addition salts can be derived include, but are not limited to, isopropylamine, benzathine, cholinate, diethanolamine, diethylamine, lysine, meglumine, piperazine and tromethaminc.

A salt (e.g., pharmaceutically acceptable salt) of a compound described herein can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

It will be understood that when the compound described herein contains more than one basic moiety or more than one acidic moiety, each such moiety can independently be involved in forming an acid addition salt form or base addition salt form, with all possible salt forms being included in this disclosure. Further, when two or more moieties of a compound are in salt form, the anions or cations forming the two or more salt forms can be the same or different. Typically, the anions or cations forming the two or more salt forms are the same. Typical molar ratios of an anion or cation in a salt of a compound of the present disclosure to a compound described herein are 3:1, 2:1, 1:1, 2:1, 3:1, 4:1 and 5:1. In some embodiments, the molar ratio of an anion or cation (e.g., anion) in a salt of a compound described herein to the compound is 1:1.

Lists of suitable salts are found in Allen, L. V., Jr., ed., Remington: The Science and Practice of Pharmacy, 22nd Edition, Pharmaceutical Press, London, UK (2012), the relevant disclosure of which is hereby incorporated by reference in its entirety.

Compounds described herein are also provided, and can be administered, as a free base.

The term “solvate” means a physical association of a compound of the present disclosure with one or more solvent molecules, whether organic or inorganic. This physical association includes hydrogen bonding. In certain instances, the solvate will be capable of isolation, for example, when one or more solvent molecules are incorporated in the crystal lattice of a crystalline solid. The solvent molecules in the solvate may be present in a regular arrangement and/or a non-ordered arrangement. The solvate may comprise either a stoichiometric or nonstoichiometric amount of the solvent molecules. “Solvate” encompasses both solution phase and solid phase solvates. Examples of solvates include, but are not limited to, hydrates, ethanolates, methanolates, and isopropanolates. Methods of solvation are generally known in the art.

A “pharmaceutically acceptable carrier” refers to media generally accepted in the art for the delivery of biologically active agents to animals, in particular, mammals, including, generally recognized as safe (GRAS) solvents, dispersion media, coatings, surfactants, antioxidants, preservatives (e.g., antibacterial agents, antifungal agents), isotonic agents, absorption delaying agents, salts, preservatives, drug stabilizers, binders, buffering agents (e.g., maleic acid, tartaric acid, lactic acid, citric acid, acetic acid, sodium bicarbonate, sodium phosphate, and the like), disintegration agents, lubricants, sweetening agents, flavoring agents, dyes, and the like, and combinations thereof, as would be known to those skilled in the art (see, for example, Allen, L. V., Jr. et al., Remington: The Science and Practice of Pharmacy (2 Volumes), 22nd Edition, Pharmaceutical Press (2012).

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

As used herein, a subject (e.g., a human) is “in need of” a treatment if such subject would benefit biologically, medically or in quality of life from such treatment. In some embodiments, the subject is over 18 years old, e.g., over 21, 30, 40, 50, 60, 65, 70, or 80 years old. In some embodiments, the subject is over 65 years old. In some embodiments, the subject is a geriatric patient.

The term “administer,” “administering,” or “administration” refers to implanting, absorbing, ingesting, injecting, inhaling, or otherwise introducing the referenced material (e.g., compound described herein, or a pharmaceutically acceptable salt thereof, or a composition thereof), in or on a subject.

The terms “treatment,” “treat,” and “treating” refer to administration of a medication or medical care to a subject, such as a human, having a disease or condition of interest, and includes: (i) preventing the disease or condition from occurring in a subject, in particular, when such subject is predisposed to the condition but has not yet been diagnosed as having it; (ii) inhibiting the disease or condition, e.g., arresting its development; (iii) relieving the disease or condition, e.g., causing regression of the disease or condition; and/or (iv) relieving the symptoms resulting from the disease or condition (e.g., pain, weight loss, cough, fatigue, weakness, etc.). Treating thus includes reversing, alleviating, delaying the onset of, and/or inhibiting the progress of a disease (e.g., a disease described herein). In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms. Treatment may also be continued after symptoms have resolved, for example, to delay or prevent recurrence.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. Alternatively, an effective amount is a prophylactically effective amount. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition, for example, an amount sufficient to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for treating in any disease or condition described.

A “prophylactically effective amount” of a compound described herein is an amount sufficient to prevent a condition, or one or more symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

As used herein, “inhibition”, “inhibiting”, “inhibit” and “inhibitor”, and the like, refer to the ability of a compound to reduce, slow, halt, or prevent the activity of a biological process (e.g., the activity of an activin receptor-like kinase (e.g., ALK-5) in a subject or cell) or change thereby the progress of a disease by, for example, altering a signaling pathway, for example, altering TGF-β1 signaling.

As will be appreciated by one of skill in the art, reference herein to “compounds of the disclosure,” “compounds described herein,” and the like refers to any compound discussed herein, as well as isomers, such as stereoisomers (including diastereoisomers, enantiomers and racemates), geometrical isomers, conformational isomers (including rotamers and astropisomers), tautomers, isotopically labeled compounds (including deuterium substitutions), and inherently formed moieties (e.g., polymorphs and/or solvates, such as hydrates) thereof. When a moiety is present that is capable of forming a salt, then salts are included as well, in particular, pharmaceutically acceptable salts. Compounds of the present disclosure can also be provided as amorphous solids or crystalline solids. Lyophilization can be employed to provide the compounds of the present disclosure as a solid. Such solid forms are also included in these terms. For example, a description using the structural representation of a free base form of a compound of the disclosure contemplates hydrates, solvates, polymorphs, co-crystals, salts, tautomers, stereoisomers, and isotopically labeled derivatives of the compounds. For example, a structural representation of a free base form of a compound of the disclosure contemplates all salt forms (e.g., pharmaceutically acceptable salt forms) of the compound. For example, a structural representation lacking stereochemical designation of a compound of the disclosure having asymmetric carbon centers contemplates all isomers, including isolation of one or more particular isomers in all levels of enantiomeric or diastereomeric purity. For example, a structural representation of a compound of the disclosure having keto/enol tautomeric forms in one particular tautomeric form contemplates all tautomeric forms of the compound.

As used herein, “tautomers” refers to compounds whose structures differ markedly in the arrangement of atoms, but which exist in easy and rapid equilibrium. It is to be understood that compounds of the present disclosure may be depicted as different tautomers. It should also be understood that when compounds have tautomeric forms, all tautomeric forms are intended to be within the scope of the disclosure, and the naming of the compounds does not exclude any tautomeric form. A tautomer is one of two or more structural isomers that exist in equilibrium and are readily converted from one isomeric form to another. This reaction results in the formal migration of a hydrogen atom accompanied by a shift of adjacent conjugated double bonds. In solutions where tautomerization is possible, a chemical equilibrium of the tautomers can be reached. The exact ratio of the tautomers depends on several factors, including temperature, solvent, and pH. The concept of tautomers that are interconvertible by tautomerizations is called tautomerism.

As used herein, an “amino acid” or “residue” refers to any naturally or non-naturally occurring amino acid, any amino acid derivative or any amino acid mimic known in the art. Included are the L- as well as the D-forms of the respective amino acids, although the L-forms are usually preferred. In some embodiments, the term relates to any one of the 20 naturally occurring amino acids: glycine (Gly), alanine (Ala), valine (Val), leucine (Leu), isoleucine (Ile), proline (Pro), cysteine (Cys), methionine (Met), serine (Ser), threonine (Thr), glutamine (Gin), asparagine (Asn), glutamic acid (Glu), aspartic acid (Asp), lysine (Lys), histidine (His), arginine (Arg), phenylalanine (Phe), tryptophan (Trp), and tyrosine (Tyr) in their L-form. In certain embodiments, the amino acid side-chain may be a side-chain of Gly, Ala, Val, Leu, Ile, Met, Cys, Ser, Thr, Trp, Phe, Lys, Arg, His, Tyr, Asn, Gln, Asp, Glu, or Pro.

The term “oligopeptide” is used to refer to a peptide with fewer members of amino acids as opposed to a polypeptide or protein. Oligopeptides described herein, are typically comprised of about two to about forty amino acid residues. Oligopeptides include dipeptides (two amino acids), tripeptides (three amino acids), tetrapeptides (four amino acids), pentapeptides (five amino acids), hexapeptides (six amino acids), heptapeptides (seven amino acids), octapeptides (eight amino acids), nonapeptides (nine amino acids), decapeptides (ten amino acids), undecapeptides (eleven amino acids), dodecapeptides (twelve amino acids), icosapeptides (twenty amino acids), tricontapeptides (thirty amino acids), tetracontapeptides (forty amino acids), etc. Oligopeptides may also be classified according to molecular structure: aeruginosas, cyanopeptolins, microcystins, microviridins, microginins, anabaenopeptins and cyclamides, etc. Homo-oligopeptides are oligopeptides comprising the same amino acid. In preferred embodiments, homo-oligopeptides comprise 10 amino acid poly-valine, poly-alanine, and poly-glycine hexamers.

The meaning of the term “peptides” are defined as small proteins of two or more amino acids linked by the carboxyl group of one to the amino group of another. Accordingly, at its basic level, peptide synthesis of whatever type comprises the repeated steps of adding amino acid or peptide molecules to one another or to an existing peptide chain. The term “peptide” generally has from about 2 to about 100 amino acids, whereas a polypeptide or protein has about 100 or more amino acids, up to a full length sequence which may be translated from a gene. Additionally, as used herein, a peptide can be a subsequence or a portion of a polypeptide or protein. In certain embodiments, the peptide consists of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues. In preferred embodiments, the peptide is from between about 30 to about 100 amino acids in length. In some embodiments, the peptide is from between about 40 to about 100 amino acids in length.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopcia or other generally recognized pharmacopcia for use in animals, and more particularly in humans.

As used herein, the term “prodrug” is intended to encompass therapeutic biologics which, under physiologic conditions, are converted into the therapeutically active biologics of the present disclosure. A common method for making a prodrug is to include one or more selected moieties which are hydrolyzed under physiologic conditions to reveal the desired molecule. In other embodiments, the prodrug is converted by an enzymatic activity of the host animal. For example, esters or carbonates (e.g., esters or carbonates of alcohols or carboxylic acids) are preferred prodrugs of the present disclosure. In certain embodiments, some or all of the molecules in a composition represented above can be replaced with the corresponding suitable prodrug, e.g., wherein a hydroxyl in the parent molecule is presented as an ester or a carbonate or carboxylic acid present in the parent therapeutic biologic is presented as an ester.

The meaning of the term “protein” is defined as a linear polymer built from about 20 different amino acids. The type and the sequence of amino acids in a protein are specified by the DNA that produces them. In certain embodiments, the sequences can be natural and unnatural. The sequence of amino acids determines the overall structure and function of a protein. In some embodiments, proteins can contain 50 or more residues. In preferred embodiments, proteins can contain greater than about 101 residues in length. A protein's net charge can be determined by two factors: 1) the total count of acidic amino acids vs. basic amino acids; and 2) the specific solvent pH surroundings, which expose positive or negative residues. As used herein, “net positively or net negatively charged proteins” are proteins that, under non-denaturing pH surroundings, have a net positive or net negative electric charge. In general, those skilled in the art will recognize that all proteins may be considered “net negatively charged proteins”, regardless of their amino acid composition, depending on their pH and/or solvent surroundings. For example, different solvents can expose negative or positive side chains depending on the solvent pH. Proteins or peptides are preferably selected from any type of enzyme or antibodies or fragments thereof showing substantially the same activity as the corresponding enzyme or antibody. Proteins or peptides may serve as a structural material (e.g. keratin), as enzymes, as hormones, as transporters (e.g. hemoglobin), as antibodies, or as regulators of gene expression. Proteins or peptides are required for the structure, function, and regulation of cells, tissues, and organs.

The term “substantially” as used herein, refers to a majority of, or mostly, as in at least about 50%, 60%, 70%, 80%, 90%, 95%, 96%, 97%, 98%, 99%, 99.5%, 99.9%, 99.99%, or at least about 99.999% or more.

It is understood that the specific order or hierarchy of steps in the methods or processes disclosed is an illustration of example approaches. Based upon design preferences, it is understood that the specific order or hierarchy of steps in the methods or processes may be rearranged. Some of the steps may be performed simultaneously. The accompanying methods claims present elements of the various steps in a sample order, and are not meant to be limited to a specific hierarchy or order presented. A phrase such as “embodiment” does not imply that such embodiment applies to all configurations of the subject technology. A disclosure relating to an embodiment may apply to all embodiments, or one or more embodiments. A phrase such as an embodiment may refer to one or more embodiments and vice-versa.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless the context requires otherwise, throughout the specification and claims that follow, the word “comprise” and synonyms and variants thereof such as “have” and “include”, as well as variations thereof, such as “comprises” and “comprising”, are to be construed in an open, inclusive sense, e.g., “including, but not limited to.” The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element or step not specified in the claim; and (iii) “consisting essentially of” limits the scope of a claim to the specified materials or steps “and those that do not materially affect the basic and novel characteristic(s)” of the claimed invention and disclosure. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system. Unless explicitly stated otherwise within the disclosure, claims, result or embodiment, “about” means within one standard deviation per the practice in the art, or can mean a range of ±20%, ±10%, ±5%, ±4, ±3, ±2 or ±1% of a given value. It is to be understood that the term “about” can precede any particular value specified herein, except for particular values used in the Examples.

All percents are intended to be weight percent (on a dry weight basis) unless otherwise specified. The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings. Thus, such other embodiments and modifications are intended to fall within the scope of the present disclosure. Further, although the present disclosure has been described herein in the context of a particular implementation in a particular environment for a particular purpose, those of ordinary skill in the art will recognize that its usefulness is not limited thereto and that the present disclosure may be beneficially implemented in any number of environments for any number of purposes. Accordingly, the claims set forth below should be construed in view of the full breadth and spirit of the present disclosure as described herein.

### EXAMPLES

### Example 1

### Health Profile Information of a Patient for Developing a Treatment Plan:

This patient is currently undergoing chemotherapy for breast cancer and reports symptoms of pain, anxiety, and significant sleep disturbances-specifically difficulty falling asleep often and staying asleep consistently. They also experience nausea that varies with their treatment cycle. Their physical functioning is impacted, especially with activities like walking, sitting, and playing with their grandkids. They are taking opioids for symptom management and have used THC in the past two months, typically around 5 mg, though they are unsure how it made them feel. While they are open to feeling intoxicated at some times of the day, maintaining mental clarity is still important to them during other parts of the day. They do not have any heart conditions, bipolar disorder, or schizophrenia.

### Example 2

### Example Treatment Plan Explanation (e.g., System-Generated Careplan Explanation)

### A) Guided Relief Capsules

### for Your Daytime Pain and Anxiety

We chose this product to be taken regularly during the day to help reduce your pain and anxiety while allowing you to stay mentally clear, especially since you want to avoid feeling high at some times of the day.

These capsules contain mostly CBD with some supporting cannabinoids, and they are meant to build up in your system over time for steady relief. We kept the dose moderate to balance symptom relief with clarity, and to stay mindful of your chemotherapy treatment and medications.

Because you are going through cancer treatment, we chose a conservative CBD dose to avoid any added pressure on your liver.

### B) Guided Comfort Tincture

### for Your Breakthrough Pain, Anxiety, or Nausea

This fast-acting tincture was chosen to help you manage sudden spikes in pain, anxiety, or nausea that can come and go with your treatment cycle.

Since you have used 5 mg THC in the past but are not sure how it felt, we started at a gentle dose that should still provide noticeable relief without being overwhelming. You can use this product when needed throughout the day or evening.

Just to note: Because you are using opioids, be aware that cannabis can increase the sedative effects of the opioids.

### C) Guided Rest Capsule

### for Your Overnight Sleep Support

We chose this bedtime product because you often have trouble falling asleep and

always have trouble staying asleep. This capsule contains CBD and CBN to support both falling and staying asleep. CBN is especially helpful for staying asleep, and the combination of cannabinoids works together in what is called the “entourage effect”-they are more effective together than alone.

This product is designed to act slowly and last through the night to reduce wake-ups.

Because you are in active cancer treatment, we were careful to stay conservative with the CBD dose here as well.

### Example 4: Example Treatment Plan

TABLE 1
Example of a treatment plan generated by a digital platform.

| | |
|---|---|
| Morning 7:30am | On days when you're not well- hydrated, |
| Onset: 15-45 minutes | you might experience lightheadedness/ |
| Duration: 2-5 hours | dizziness. This is not a concern, and can |
| | usually be remedied by drinking a large |
| | glass of water. The experience of |
| | grogginess or discomfort on the day |
| | following cannabis use is often related to |
| | dehydration. |
| | Directed |
| | Take 2 Capsules (133 mg Cannabidiol |
| | (CBD)/33 mg Cannabichromene (CBC)) of |
| | Guided Relief Capsules |
| | Swallow 2 Capsules (133 mg CBD/33 mg |
| | CBC) directly while drinking a glass of |
| | water. |
| Afternoon 1:00pm | On days when you're not well-hydrated, |
| Onset: 15-45 minutes | you might experience |
| Duration: 2-5 hours | lightheadedness/dizziness. This is not a |
| | concern, and can usually be remedied by |
| | drinking a large glass of water. The |
| | experience of grogginess or discomfort on |
| | the day following cannabis use is often |
| | related to dehydration. |
| | Directed |
| | Take 2 Capsules (133 mg CBD/33 mg |
| | CBC) of Guided Relief Capsules |
| | Swallow 2 Capsules (133 mg CBD/33 mg |
| | CBC) directly while drinking a glass of |
| | water. |
| Evening 6:30pm | The recommended products contain THC; |
| Onset: 15-45 minutes | you may feel somewhat altered or a sense |
| Duration: 2-5 hours | of euphoria. Please use caution or avoid |
| | doing safety sensitive activities (like |
| | driving) until you know how cannabis |
| | impacts your ability to feel clear and |
| | function safely. This learning may take |
| | days to weeks. |
| | On days when you're not well-hydrated, |
| | you might experience light |
| | headedness/dizziness. This is not a concern, |
| | and can usually be remedied by drinking a |
| | large glass of water. The experience of |
| | grogginess or discomfort on the day |
| | following cannabis use is often related to |
| | dehydration. |
| | Directed |
| | Take 2 Pumps (3 mg Tetrahydrocannabinol |
| | (THC)) of Guided Comfort Tincture |
| | Take 2 Pumps (3 mg THC) under the |
| | tongue. Hold the drops there for at least 90 |
| | seconds before swallowing completely. |
| Bedtime 9:00pm | On days when you're not well- hydrated, |
| Onset: 15-45 minutes | you might experience |
| Duration: 2-5 hours | lightheadedness/dizziness. This is not a |
| | concern, and can usually be remedied by |
| | drinking a large glass of water. The |
| | experience of grogginess or discomfort on |
| | the day following cannabis use is often |
| | related to dehydration. |
| | Directed |
| | Take 1 Capsule (12.5 mg CBD/2.5 mg |
| | Cannabinol (CBN)) of Guided Rest Capsules |
| | Swallow 1 Capsule (12.5 mg CBD/2.5 mg |
| | CBN) directly while drinking a glass of |
| | water. |

### Example 5: Treatment Adherence Data

TABLE 2
Cannabinoid-based treatment adherence data was collected
over 26 weeks from various subjects. The number
of responses is indicated in the ‘n’ column.

| | | | Most of | | |
|---|---|---|---|---|---|
| | Never | Sometimes | the Time | Always | n |
| Week 1 | 3% | 20% | 35% | 42% | 66 |
| Week 2 | 0% | 16% | 39% | 45% | 31 |
| Week 4 | 4% | 17% | 39% | 39% | 23 |
| Week 6 | 0% | 17% | 56% | 28% | 18 |
| Week 8 | 0% | 0% | 46% | 54% | 13 |
| Week 10 | 0% | 11% | 22% | 67% | 9 |
| Week 12 | 0% | 22% | 11% | 67% | 9 |
| Week 14 | 0% | 0% | 33% | 67% | 9 |
| Week 16 | 0% | 8% | 46% | 46% | 13 |
| Week 18 | 0% | 0% | 73% | 27% | 11 |
| Week 20 | 0% | 0% | 73% | 27% | 11 |
| Week 22 | 0% | 9% | 64% | 27% | 11 |
| Week 24 | 0% | 13% | 50% | 38% | 8 |
| Week 26 | 0% | 0% | 50% | 50% | 6 |
| TOTALS | 1% | 13% | 43% | 43% | |

### Example 6

Cohen's d Test & Statistical Significance by Patient Important Outcomes (PIOs) Category data was collected from subjects administered cannabinoid-based products of the present disclosure based on personalized treatment plan (i.e., treatment plan based on subject's data). Table 3 shows the improvement of subjects determined by PIO outcomes.

Effect sizes are categorized as no effect (<0.2), a small effect (>=0.2), a medium effect (>=0.5), or a large effect (>=0.8). A larger effect size indicates that the improvement is likely meaningful for patients, but any effect size indicates some improvement. Statistical significance was calculated using a p value of 0.05.

TABLE 3

| | | | | Activities | |
|---|---|---|---|---|---|
| | | | | Related | |
| | | General | Activities | to Work, | |
| | | Activities | of | Social, or | Disease- |
| | All | and | Daily | Leisure | Specific |
| | categories | Mobility | Living | Time | Symptoms |
| Effect Size | large | large | large | large | large |
| d-score | 1.04 | 1.14 | 1.69 | 0.87 | 1.39 |
| Statistical | TRUE | TRUE | TRUE | TRUE | TRUE |
| Significance | | | | | |
| p-value | 0.0000 | 0.0000 | 0.0001 | 0.0002 | 0.0010 |
| | (n = 114) | (n = 46) | (n = 13) | (n = 46) | (n = B) |

General activities and mobility: Broad tasks or actions a person might do across any part of their life—at home, in the community, or during any time of day, but which do not fall neatly into self-care, work, or leisure categories. They reflect basic physical functioning. Examples: walking, standing, exercise.

Daily living activities: These are essential self-care tasks required to live independently and safely. They are often used in clinical assessments to evaluate a person's functional status. Examples: getting dressed, brushing teeth, sitting at the table for a meal, household chores.

Activities related to work, social, or leisure time: These activities involve participation in structured or unstructured roles beyond self-care and basic tasks. They reflect quality of life, social engagement, and productivity. Examples: spending time with family, working, gardening, painting.

Regarding reported intolerable side effects, the results are as follows.

TABLE 4

| | |
|---|---|
| Side Effects Rapidly Resolved | 6.60% |
| Percentage of Patients with more than 2 | 5.63% |
| consecutive weeks of side effects | |

Percentage of patients presented with meaningful improvement are presented as follows.

TABLE 5

| | | | | Gastrointestinal |
|---|---|---|---|---|
| | Pain | Sleep | | Nausea and |
| | Interference | Disturbance | Anxiety | Vomiting |
| After 4 weeks | 68% | 75% | 83% | 91% |
| of care | (n = 36) | (n = 36) | (n = 32) | (n = 12) |
| After 12 weeks | 93% | 77% | 100% | 100% |
| of care | (n = 16) | (n = 14) | (n = 11) | (n = 2) |

### Example 7

PROMIS® metric scores across four weeks were collected from cancer patients (see FIG. 7) administered cannabinoid-based products of the present disclosure based on personalized treatment plan.

### Embodiments

1. A cannabinoid-based composition for therapeutic use in older adults, comprising:
   a) Cannabinoids sourced from cannabis or hemp plants;
   b) Advanced functional ingredients selected for their synergistic effects with cannabinoids in alleviating chronic pain, inflammation, cognitive decline, and sleep disturbances associated with aging;
   c) Nutritional supplements chosen to support overall health and well-being in older adults.
2. The composition of Embodiment 1, wherein the cannabinoids and advanced functional ingredients are present in specific ratios optimized for targeting chronic pain, inflammation, cognitive decline, and sleep disturbances in older adults.
3. A method for formulating cannabinoid-based products tailored to the specific health needs of older adults, comprising:
   a) Identifying key symptoms and concerns commonly experienced by older adults dealing with chronic pain, inflammation, cognitive decline, and sleep disturbances;
   b) Selecting cannabinoids, advanced functional ingredients, and nutritional supplements based on their therapeutic properties and compatibility with aging-related conditions;
   c) Formulating the composition in advanced non-combustible form factors to ensure safe and consistent delivery of active ingredients.
4. The method of Embodiment 3, further comprising:
   a) Integrating an advanced patient digital platform utilizing AI algorithms for personalized dosing regimens and remote monitoring of therapeutic outcomes for older adult consumers;
   b) Adjusting treatment plans in real-time based on individual responses and feedback to optimize therapeutic outcomes and enhance patient adherence.
5. A non-combustible delivery device for administering cannabinoid-based compositions to older adults, comprising:
   a) A delivery system configured to administer the composition in advanced non-invasive form factors suitable for older adults, such as transdermal patches, controlled-release capsules, or other innovative delivery methods;
   b) A controlled dosing mechanism to regulate the amount of active ingredients delivered to the patient;
   c) Safety features to prevent misuse and ensure user compliance with recommended dosing protocols.
6. A cannabinoid-based composition for therapeutic use in cancer patients, comprising:
   a) Cannabinoids sourced from cannabis or hemp plants;
   b) Functional ingredients selected for their synergistic effects with cannabinoids in alleviating symptoms commonly experienced by cancer patients;
   c) Nutritional supplements chosen to support overall health and well-being during cancer treatment.
7. The composition of Embodiment 6, wherein the cannabinoids are present in a specific ratio optimized for targeting symptoms such as impaired appetite, weight loss, chronic pain, nausea, and insomnia in cancer patients.
8. A method for formulating cannabinoid-based products tailored to the specific health outcomes of cancer patients, comprising:
   a) Identifying key symptoms and side effects commonly experienced by cancer patients undergoing treatment;
   b) Selecting cannabinoids, functional ingredients, and nutritional supplements based on their therapeutic properties and compatibility with cancer treatment regimens;
   c) Formulating the composition in non-combustible form factors to ensure safe and consistent delivery of active ingredients.
9. The method of Embodiment 8, further comprising:
   a) Integrating a patient digital platform to create personalized regimens and dosing instructions based on individual patient characteristics and treatment goals;
   b) Monitoring patient responses and adjusting treatment plans in real-time to optimize therapeutic outcomes and minimize adverse effects.
10. Non-combustible delivery device for administering these unique cannabinoid-based compositions to cancer patients, comprising:
   a) An inhaler and/or vaporization system configured to heat the composition to a temperature suitable for vaporization without combustion;
   b) A controlled dosing mechanism to regulate the amount of active ingredients delivered to the patient;
11. The delivery device of Embodiment 10, further comprising:
   a) A user interface integrated with the patient digital platform to provide real-time feedback and guidance on usage, dosing, and treatment adherence;
   b) Wireless connectivity for remote monitoring by healthcare providers and researchers to track patient progress and outcomes.
12. Cannabinoid-based products enhanced by a model-based care regimen generator for optimizing therapeutic effects, comprising: a. Formulations of cannabinoid-based products specifically targeting chronic pain, cancer-related symptoms, aging-related challenges, sleep disturbances, and anxiety; b. A model-based care regimen generator utilizing advanced algorithms to analyze patient data and generate personalized treatment plans tailored to individual patient characteristics and treatment goals.
13. The cannabinoid-based products of Embodiment 12, wherein the formulations incorporate cannabinoids, advanced functional ingredients, and nutritional supplements in precise ratios optimized for therapeutic efficacy.
14. A method for optimizing therapeutic effects of cannabinoid-based products using a model-based care regimen generator, comprising: a. Analyzing patient data to identify individual health profiles, symptom severity, and treatment responses; b. Generating personalized treatment plans outlining specific combinations of cannabinoid-based products based on patient characteristics and treatment goals; c. Monitoring patient-reported outcomes and adherence data to refine treatment protocols and optimize therapeutic effects over time.
15. The method of Embodiment 14, further comprising: a. Providing patients with access to a user-friendly digital platform for real-time feedback, symptom tracking, and communication with healthcare providers; b. Leveraging predictive analytics to identify trends, patterns, and potential interventions for further enhancing therapeutic efficacy and patient satisfaction.
16. A computer-readable medium storing instructions for executing the method of Embodiment 14, wherein the instructions comprise code for analyzing patient data, generating personalized treatment plans, monitoring patient-reported outcomes and adherence data, and optimizing treatment protocols in real-time.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments.

1. A method of improving an outcome of a subject, the method comprising:
administering a composition comprising a cannabinoid to the subject based on a treatment plan that is based on the subject's data, the cannabinoid intended to cause an improved outcome of the subject, wherein the outcome comprises one or more of: (i) reducing a condition of the subject, (ii) improving mobility of the subject and reducing difficulty of performing general activities by the subject, (iii) reducing difficulty of performing daily living activities by the subject, or (iv) reducing difficulty of performing activities related to work, social, or leisure time by the subject.

2. The method of claim 1, wherein reducing the condition of the subject comprises reducing one or more symptoms associated with a disease or a therapy.

3. The method of claim 2, wherein the disease is cancer, and wherein the therapy is chemotherapy.

4. The method of claim 1, wherein the condition of the subject comprises chronic pain, inflammation, cancer symptoms, sleep disturbances, anxiety, or a combination thereof.

5. The method of claim 1, wherein the treatment plan is provided by a digital platform.

6. The method of claim 5, wherein the digital platform comprises a model-based treatment plan generator.

7. The method of claim 1, wherein the subject's data comprises:
a health profile of the subject comprising one or more of: severity of the condition of the subject, the mobility of the subject and the difficulty of performing general activities by the subject, the difficulty of performing daily living activities by the subject, or the difficulty of performing activities related to work, social, or leisure time by the subject; and
treatment goals of the subject.

8. A method of reducing pain, sleep disturbance, anxiety, or gastrointestinal nausea and vomiting in a subject, the method comprising: administering a composition comprising a cannabinoid to the subject.

9. The method of claim 8, wherein the composition comprising the cannabinoid is administered to the subject based on a treatment plan.

10. The method of claim 9, wherein the treatment plan is: based on the subject's data; and provided by a digital platform comprising a model-based treatment plan generator.

11. The method of claim 10, wherein the subject's data comprises:
a health profile of the subject comprising severity of pain, sleep disturbance, anxiety, or gastrointestinal nausea and vomiting of the subject; and
treatment goals of the subject.

12. (canceled)

13. The method of claim 1, wherein the composition is a first composition and the cannabinoid is a first cannabinoid, and wherein the method further comprises: administering a second composition comprising the first cannabinoid to the subject.

14. The method of claim 13, wherein the second composition comprising the first cannabinoid is administered to the subject based on an updated treatment plan.

15. The method of claim 14, wherein the updated treatment plan is based on updated subject's data and provided by a digital platform comprising a model-based treatment plan generator.

16. The method of claim 15, wherein the updated subject's data comprises one or more of: feedback from the subject, or treatment adherence data.

17. The method of claim 13, wherein the first cannabinoid is tetrahydrocannabinol, tetrahydrocannabivarin, or a combination thereof.

18. The method of claim 13, wherein the first composition further comprises a second cannabinoid.

19. The method of claim 13, wherein the second composition further comprises a second cannabinoid.

20. The method of claim 18, wherein the second cannabinoid is cannabigerol, cannabichromene, cannabinol, delta-8-tetrahydrocannabinol, tetrahydrocannabinolic acid, cannabidiolic acid, or a combination thereof.

21. A composition, comprising a first cannabinoid, a second cannabinoid, and an agent, wherein: the agent comprises protein, amino acids, or a combination thereof; the first cannabinoid is cannabidiol; the second cannabinoid is tetrahydrocannabivarin or delta-9-tetrahydrocannabinol; and the composition has a mass ratio of a combined mass of the first cannabinoid and the second cannabinoid to the agent that is between about 1:10 and about 1:1.

22. (canceled)

23. (canceled)

24. (canceled)

25. (canceled)

26. (canceled)

27. (canceled)

28. (canceled)
